(19) United States
(12) Patent Application Publication
(10) Pub. No.: US 2013/0053438 A1
(43) Pub. Date: Feb. 28, 2013

(54) PHARMACEUTICAL COMPOSITIONS COMPRISING LIGNANS AND THEIR DERIVATIVES FOR THE MEDICAL MANAGEMENT OF ANGIOGENESIS AND HYPOVASCULARITY

(71) Applicants: David Bernhard, Wien (AT); Hermann Stuppner, Gotzens (AT); Stefan Schwaiger, Innsbruck (AT); Dominik Wiedemann, Hall in Tirol (AT); Gerold Untergasser, Innsbruck (AT)

(72) Inventors: David Bernhard, Wien (AT); Hermann Stuppner, Gotzens (AT); Stefan Schwaiger, Innsbruck (AT); Dominik Wiedemann, Hall in Tirol (AT); Gerold Untergasser, Innsbruck (AT)

(73) Assignees: Universitat Innsbruck, Innsbruck (AT); Medizinische Universitat Wien, Wien (AT)

(21) Appl. No.: 13/521,016

(22) PCT Filed: Jan. 19, 2011
(86) PCT No.: PCT/EP11/50701

§ 371 Date: Jul. 6, 2012

Related U.S. Application Data
(60) Provisional application No. 61/336,288, filed on Jan. 19, 2010.

(30) Foreign Application Priority Data
Jan. 19, 2010 (EP) 10151086.5

(51) Int. Cl. A61K 31/341; A61P 9/00; A61P 17/00; A61P 9/10; A61P 27/02; A61P 15/10; A61P 7/02; A61P 9/08; A61P 11/00; C07D 307/16; A61P 25/28

(52) U.S. Cl. 514/461; 549/501

(57) ABSTRACT

The present invention relates to a pharmaceutical composition for stimulating angiogenesis and/or the treatment or prevention of hypovascularity and/or the prevention and/or treatment of an angiogenic disorder/disease, whereby the composition comprises specific compounds which may be obtained from Leontopodium alpinum Cass, (Edelweiss). These compounds relate to lignan compounds as shown in herein disclosed formula I. A preferred compound in this context is leologin—IUPAC name [(2S,3R,4R)-4-(3,4-dimethoxybenzyl)-2-(3,4-dimethoxyphenyl)tetxahydrofuran-3-yl]methyl (2Z)-2-methylbut-2-enoat], and even more particularly 5-methoxy-leoligin (IUPAC name: [(25,3R,4R)-4-(3,4-dimethoxybenzyl)-2-(3,4,5-trimethoxyphenyl)tetrahydrofuran-3-yl]methyl-(2Z)-2-methylbut-2-enoat) and derivatives thereof. Corresponding means and methods in respect of medical uses of these compounds are described. The compounds provided herein may particularly be useful in the treatment of wound healing, in particular traumatic wounds (like, but not limited to surface and skin wounds), non-diabetic retinopathy, vascular obliteration. The compounds derived from Leontopodium alpinum Cass. (Edelweiss) as described herein are also useful in the re-vascularization of tissue after amputation as well during or after transplantation of tissues or organs. These compounds are also useful in the medical intervention of arterio- and veno-microvasculopathy of blood vessels, in particular retinal microvasculopathy, arterio- and veno-microangiopathy that preferably cannot be treated by surgery, in ischemic diseases or ischemic disorders or in the treatment or prevention of necrosis/necrotic events, in particular of ischemic diseases or necrosis/necrotic events that cannot be treated by surgery. These compounds may also be used in the treatment or prevention of stable angina abdominalis, vascular dementia, impotence or penile dysfunction and the like and they may be employed in the reactivation of necrotisising tissue or in the reactivation of hibernating tissue.

The present invention relates to a pharmaceutical composition for stimulating angiogenesis and/or the treatment or prevention of hypovascularity and/or the prevention and/or treatment of an angiogenic disorder/disease, whereby the composition comprises specific compounds which may be obtained from Leontopodium alpinum Cass. (Edelweiss). These compounds relate to lignan compounds as shown in herein disclosed formula I. A preferred compound in this context is leologin—IUPAC name [(2S,3R,4R)-4-(3,4-dimethoxybenzyl)-2-(3,4-dimethoxyphenyptetrahydrofuran-3-yl]methyl (2Z)-2-methylbut-2-enoat], and even more particularly 5-methoxy-leoligin (IUPAC name: [(2S,3R,4R)-4-(3,4-dimethoxybenzyl)-2-(3,4,5-trimethoxyphenyptetrahydrofuran-3-yl]methyl-(2Z)-2-methylbut-2-enoat) and derivatives thereof. Corresponding means and methods in respect of medical uses of these compounds are described. The compounds provided herein may particularly be useful in the treatment of wound healing, in particular traumatic wounds (like, but not limited to surface and skin wounds), non-diabetic retinopathy, vascular obliteration.

The compounds derived from Leontopodium alpinum Cass. (Edelweiss) as described herein are also useful in the re-vascularization of tissue after amputation as well during or after transplantation of tissues or organs. These compounds are also useful in the medical intervention of arterio- and veno-microvasculopathy of blood vessels, in particular retinal microvasculopathy, arterio- and veno-microangiopathy that preferably cannot be treated by surgery, in ischemic diseases or ischemic disorders or in the treatment or prevention of necrosis/necrotic events, in particular of ischemic diseases or necrosis/necrotic events that cannot be treated by surgery. These compounds may also be used in the treatment or prevention of stable angina abdominalis, vascular dementia, penile dysfunction (i.e. impotence) and the like and they may be employed in the reactivation of necrotisising tissue or in the reactivation of hibernating tissue.

The use of therapeutic angiogenesis (i.e. the stimulation of angiogenesis) is aimed at in various diseases. However, the available compounds and medicaments do not efficiently and reliably stimulate angiogenesis, and thus fail to supply the affected tissue or organs with nutrients and oxygen. The known pro-angiogenic vascular endothelial growth factor (VEGF) is not believed to be suitable for clinical application due to known side-effects. The only market authorization in this field has been granted for Becaplermin (platelet-derived growth factor, PDGF) for treating ulcer in the U.S.A. in 1997. Accordingly, the provision of improved therapies in this field is highly desirable.

Lignans are considered as potential candidate molecules which may be used in the treatment of diseases/disorders associated with the cardiovascular system. However, only a limited number of publications have reported on the impact of lignans on the cardiovascular system in general, and only a few different lignans have been tested so far. Some data suggest that lignans are cardiovascular protective agents.

Recently, specific lignans have been isolated from Edelweiss roots. Edelweiss root extracts show a complex pattern of secondary plant metabolites, of several compound classes like coumarins, lignans, sesquiterpenes, polyacetylens, diterpenes, and others; see Schwaiger, Planta Med, 70(10), 978-85 (2004). In general, lignans are polyphenolic plant metabolites derived from phenylalanine, which are synthesized by the coupling of two phenylpropanoid units by a bond between the β-positions in the propane side chains. One of these lignans which has been isolated from the roots of Edelweiss is leoligin—IUPAC name [(2S,3R,4R)-4-(3,4-dimethoxybenzyl)-2-(3,4-dimethoxyphenyl)tetrahydrofuran-3-yl]methyl (2Z)-2-methylbut-2-enoat].

Edelweiss (Leontopodium alpinum Cass.), one of the most popular alpine plants, has been used in folk medicine for the treatment of diarrhoea, fever, and “abdominal aches”. However, it is of note that only the upper parts (i.e. flowers, leaves and stems) of the Edelweiss plant have been used in folk medicine because these contain the bulk of the biomass and have thus been easier available. Historical references from the year 1582 mention that Edelweiss and its relatives are mainly used for the treatment of diarrhoea and dysentery; see Tabernaemontanus, J. T. (1582): “Das Ander Buch von Kreutern”. In: Bauhin, H. (ed.) (1731): D. Jacobi Theodori Tabernaemontani Neu Vollkommen Kraeuter-Buch. Reprint Basel, König, 1731. Verlag Kolbl, Grunwald (Munchen) 1993). Further information on the traditional use of Edelweiss was collected by several diploma theses on the usage of Alpine plants in folk medicine, performed at the Institute of Pharmacognosy of the University of Vienna.

Interviews of elder inhabitants of alpine regions in Austria and Northern Italy revealed a variety of local knowledge. In Vorarlberg Edelweiss flower heads were boiled in milk, preparations of which were used for the therapy of abdominal aches and diarrhoea in humans, and particularly also in domestic stock; see Kiene, “Volksmedizin in verschiedenen Gebieten Vorarlberg”, Master Thesis at the University of Vienna (1992); Bitschnau, Arzneidrogen der Volksmedizin im Montafon, Master Thesis at the University of Vienna (1991).

Similar information was also obtained for North-Tyrol, East-Tyrol and South-Tyrol, where Edelweiss was, furthermore, used to cure tonsillitis, angina and bronchitis, and as an antipyretic to lower fever; see Knechtl, “Volksmedizinisch verwendete Heilpflanzen und Hausmittel im Inntal und umgebenden Seitentälern (Tirol)”, Master Thesis at the University of Vienna (1992); Wieser, “Volksmedizinische Verwendung von Heilpflanzen und Hausmitteln im Osttiroler Pustertal mit Seitentälern und im Lesachtal”, Master Thesis at the University of Vienna (1995); Pickl-Herck, Volksmedizinische Anwendung im Norden Südtirols. Master Thesis at the University of Vienna (1995).

In Polish traditional medicine, L. alpinum was used for the therapy of breast cancer by local application of a poultice of the aerial plant parts; see Hartwell, J. Nat. Prod. (Lloydia) 31, 71-170 (1968). Knechtl (1992; loc. cit.) also describes that infusions of edelweiss flowers can be used to ameliorate stomach-ache. In particular diarrhoea in children is to be treated with milk in which flowers from edelweiss plants has been boiled; see Knechtl (1992; loc. cit.). Wieser (1995; loc. cit.) points out that upper parts of edelweiss plants are used in folk medicine, since edelweiss plants are selected out of the cut grass of alpine meadows (i.e. the upper parts of edelweiss plants are collected) and dried. This has been particularly described for the Villgratental in Kalkstein (1650 m above sea level). The edelweiss plant is described in Wieser (1995; loc. cit.) as the “camomile” of the Alps, since it is used in medicine similar to camomile. According to Wieser (1995; loc. cit.) an edelweiss infusion is used to ameliorate stomach ache, while edelweiss boiled in milk is helpful in abdominal cramping.

Pickl-Herk (1995; loc. cit.) describes the following medical use of edelweiss flowers: infusion of flowers is beneficial in ameliorating stomach ache (in particular caused by foul drinking water), stomach flatulencies, and diarrhoea with vomiting. Again, infusions of edelweiss flowers are intended to be administered in particular to children. “Edelweissmilch” (i.e. 4-5 flowers boiled in 0.5 l milk) is used for the following disorders: diarrhea, vertigo, poisoning (leads to vomiting), snake-bites, blood poisoning, indigestion, abdominal cramping, stomach ache, stomach flatulencies, or hangover; see Pickl-Herk (1995; loc. cit.). Also the use of “Edelweissmilch” in veterinary medicine is disclosed in this document, i.e. the treatment of calves suffering from diarrhoea and of calves/cows suffering from stomach flatulencies is described. Antibacterial and anti-inflammatory activity of extracts of Edelweiss have been described recently; see Dobner, J Ethnopharm 89, 301-303 (2003), Speroni, J Ethnopharm 105, 421-426 (2006) and Dobner, Planta Med 70, 502-508 (2004).

The group around Hermann Stuppner and Stefan Schwaiger has isolated, identified and quantified in form of analytical assessments of herb extracts several constituents of “Edelweiss”, see Stuppner (2002), Hevetica Chimica Acta 85, 2982-2989; Dobner (2003), Helvetica Acta 86, 733 and following; Schwaiger (2006) Phytochem. Analysis 17, 291-298 or Schwaiger (2005) Tetrahedron 61, 4621-4630. Costa and colleagues (2008) detected a chemo-protective action mechanism of leontopodic acid against aflatoxin B1 and deoxynivalenol-induced cell damage; see Costa (2008), J. Applied Toxicology 29, 7-14.

Some constituents of Edelweiss, in particular bisabolane derivates, one lignan and ent-kaurenoate were shown to posses ex vivo leukotriene biosynthesis inhibition. Scientists, including Stefan Schwaiger, Hermann Stuppner and David Bernhard, have shown that leoligin inhibits intimal hyperplasia of venous bypass grafts; see Reisinger (2009), Cardiovascular Research 82, 542-549.

A large number of lignan-based cancer therapy studies (in vitro and in vivo) showed profound cytotoxicity and cell death induction by these compounds, see Kim Planta Med, 68(3), 271-4 (2002) and Lin J Cell Biochem 84(3), 532-44 (2002). Cell-death and cytotoxicity, is however, an undesired event in a plurality of medical interventions.

Thus, the technical problem underlying the present invention is the provision of means and methods for the medical interference in diseases and disorders associated with hypovascularity and/or the provision of means and methods for the stimulation of angiogenesis.

The technical problem is solved by provision of the embodiments characterized in the claims and described herein below.

Accordingly, the present invention relates to a pharmaceutical composition comprising a compound of formula (I)

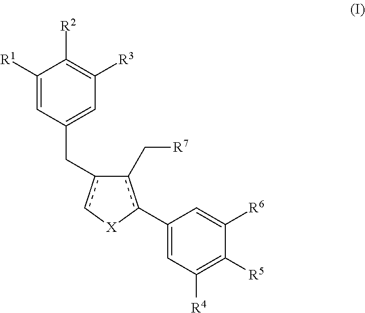

for stimulating angiogenesis and/or the prevention and/or treatment of an angiogenic disorder,
wherein
R¹, R² and R³ are independently selected from H, OH, halogen, alkyl, or alkoxy; and
R⁴, R⁵ and R⁶ are independently selected from H, OH, halogen, alkyl, or alkoxy;
R⁷ is selected from —OR⁸, —N(R^{8′})R⁸, —SR⁸, —C(O)R⁸, —OC(O)R⁹, —C(O)OR⁹, —N(R^{9′})C(O)R⁹, —C(O)N(R^{9′})R⁹ or —S(O)R⁹; wherein R⁸ and R⁹ are independently selected from alkyl or alkenyl and R^{8′} and R^{9′} are independently selected from H, alkyl or alkenyl; and wherein any alkyl or alkenyl group comprised in R⁷ may be unsubstituted or substituted by one or more substituents, selected from OH, halogen or alkoxy; and
X is selected from O, S, C(R¹⁰)R¹⁰ and NR¹⁶, wherein R¹⁰, independently for each occurrence, is H, alkyl or alkenyl;
and the dashed lines in the ring structure containing the group X indicate that the respective bond may be a single or a double bond;
or any pharmaceutically acceptable salt or solvate thereof.

In a preferred embodiment, the compound of formula (I) comprised in the pharmaceutical composition has the stereochemistry indicated in formula (Ia):

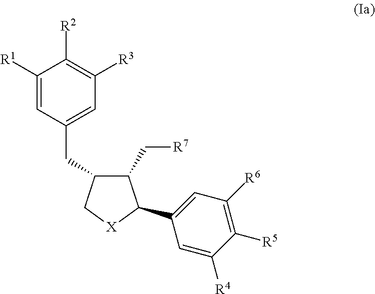

wherein R¹ to R⁷ and X are defined as described herein above.

For the above formulae (I) and (Ia), the following embodiments are preferred in the context of the invention.

Alkyl substituents, as they may be present as R¹ to R⁶, are preferably C1 to C6 alkyl groups, more strongly preferred are C1 to C3 alkyl groups, and further preferred is methyl.

Halogen substituents include fluoro-, chloro-, bromo- and iodo-atoms, with preference given to chloro and bromo.

As set out above, X is selected from O, S, C(R¹⁰)R¹⁰ and NR¹⁰; wherein R¹⁰, independently for each occurrence, is H, alkyl or alkenyl. Preferred as alkyl group is a C1 to C6 alkyl group, particularly preferred are methyl and ethyl. Preferred as an alkenyl group is a C2 to C6 alkenyl group.

Preferably, X is O or NR¹⁰, and particularly preferred is O. Preferred groups R¹⁰ are H and C1 to C6 alkyl, particularly preferred are H and methyl.

As further explained above, the dashed lines in the ring structure containing group X indicates that the respective bond may be a single or a double bond. The ring structure may contain no double bond, one double bond or two double bonds at the respective position.

Preferred are cases where no double bond is present, i.e. the ring structure containing group X is a saturated ring.

It is generally preferred that at least one of R¹ to R³ represents an alkoxy group, and it is more preferred that two or all three of them represent an alkoxy group. Among suitable alkoxy groups, general preference is given to C1 to C6 alkoxy groups, more strongly preferred are C1 to C3 alkoxy groups and particular preference is given to methoxy groups. If two of R¹ to R³ represent an alkoxy group, it is preferred that one of them is R².

In the preferred compounds referred to above, wherein one or two of R¹ to R³ represent an alkoxy group, it is further preferred that the remaining groups of R¹ to R³ represent H or an alkyl group, preferably H. Preferred alkyl groups are C1 to C6 alkyl groups, more strongly preferred are C1 to C3 alkyl groups, and further preferred is methyl.

It is generally preferred that at least one of R⁴ to R⁶ represents an alkoxy group, and it is more preferred that two or all three of them represent an alkoxy group. Among suitable alkoxy groups, general preference is given to C1 to C6 alkoxy groups, more strongly preferred are C1 to C3 alkoxy groups and particular preference is given to methoxy groups. If two of R⁴ to R⁶ represent an alkoxy group, it is preferred that one of them is R⁵.

In the preferred compounds referred to above, wherein one or two of R⁴ to R⁶ represent an alkoxy group, it is further preferred that the remaining groups of R⁴ to R⁶ represent H or an alkyl group, preferably H. Preferred alkyl groups are C1 to C6 alkyl groups, more strongly preferred are C1 to C3 alkyl groups, and further preferred is methyl.

Thus, particularly preferred are compounds wherein four, five or all six of R¹ to R⁶ are alkoxy, and the remaining groups of R¹ to R⁶, if any, are hydrogen. Mention may be made in this respect specifically of compounds wherein R¹ is H and R² and R³ are alkoxy, or all of R¹ to R³ are alkoxy; and wherein R⁴ is H and R⁵ and R⁶ are alkoxy, or all of R⁴ to R⁶ are alkoxy. Among suitable alkoxy groups, general preference is given to C1 to C6 alkoxy groups, more strongly preferred are C1 to C3 alkoxy groups and particular preference is given to methoxy groups.

R⁷ is preferably —OC(O)R⁹, —C(O)OR⁹, —N(R^{9′})C(O)R⁹, —C(O)N(R^{9′})R⁹ or —S(O)R⁹, i.e. an ester, amide or sulfoxy group, with a particular preference for the ester groups —OC(O)R⁹ or —C(O)OR⁹. Most preferred as R⁷ is a group —OC(O)R⁹.

R⁸ is preferably an alkyl or alkenyl group which is unsubstituted. Preferred alkyl groups have 2 or more, particularly 3 or more carbon atoms. It is further preferred that they have 14 or less, such as 10 or less, particularly 8 or less or 6 or less carbon atoms. Preferred alkenyl groups have 3 or more carbon atoms. It is further preferred that they have 14 or less, such as 10 or less, particularly 8 or less or 6 or less carbon atoms. Independent of the number of carbon atoms, it is preferred that the alkenyl groups have one C—C double bond.

R⁸ is preferably H or any alkyl group having 10 or less, such as 8 or less, preferably 6 or less carbon atoms, such as methyl, ethyl, or propyl.

R⁹ is preferably an alkyl or alkenyl group which is unsubstituted. Preferred alkyl groups have 2 or more, particularly 3 or more carbon atoms. It is further preferred that they have 14 or less, such as 10 or less, particularly 8 or less or 6 or less carbon atoms. Preferred alkenyl groups have 3 or more carbon atoms. It is further preferred that they have 14 or less, such as 10 or less, particularly 8 or less or 6 or less carbon atoms. Independently of the number of carbon atoms, it is preferable that the alkenyl groups have one C—C double bond. Particularly preferred as R⁹ is a branched alkenyl group as it occurs in leoligin of the formula —C(CH₃)CH—CH₃. In this group, the methyl substituents at the double bond may be in E- or Z-configuration with respect to each other, with preference for the Z-configuration.

R^{9′} is preferably H or any alkyl group having 10 or less, such as 8 or less, preferably 6 or less carbon atoms, such as methyl, ethyl, or propyl.

In a strongly preferred embodiment, the present invention concerns pharmaceutical compositions comprising compounds of formula (1) or (1a) wherein X is O; wherein, in the case of formula (1), the ring structure containing X has no double bonds; wherein four, five or all six of R¹ to R⁶ are alkoxy, and the remaining groups of R¹ to R⁶, if any, are hydrogen;

R⁷ is —OC(O)R⁹ or

—C(O)OR⁹, particularly —OC(O)R⁹; and R⁹ is an unsubstituted alkenyl group having one double bond and 8 or less carbon atoms or an unsubstituted alkyl group having two or more and 8 or less carbon atoms.

A preferred embodiment relates to a pharmaceutical composition, wherein the compound of formula (I) has the following structure:

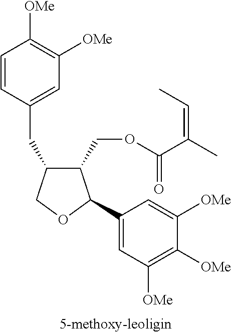 [SMILES: C/C=C(/C)C(=O)OC[C@H]1[C@@H](Cc2ccc(OC)c(OC)c2)CO[C@@H]1c1cc(OC)c(OC)c(OC)c1]

A structural formula of a further exemplary (di)methoxy-derivative of leoligin, which also represents a preferred compound in the context of the present invention, is given herein below:

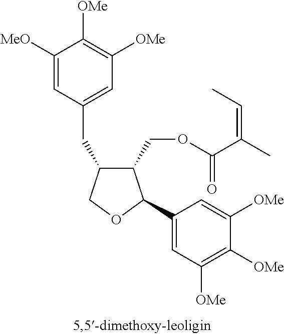 [SMILES: C/C=C(/C)C(=O)OC[C@H]1[C@@H](Cc2cc(OC)c(OC)c(OC)c2)CO[C@@H]1c1cc(OC)c(OC)c(OC)c1]

Also preferred herein is a compound of formula (I) having the following structure:

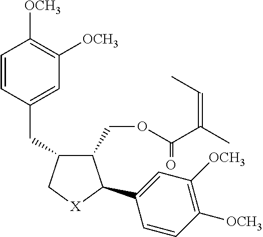

The chemical structure given herein above is (2S,3R,4R)-4-(3,4-dimethoxybenzyl)-2-(3,4-dimethoxyphenyptetrahydrofuran-3-yl]methyl (2Z)-2-methylbut-2-enoat] also known under the trivial name “leoligin”.

In particular 5-methoxy-leoligin has been shown and exemplified in the appended examples as a potent activator of angiogenesis. 5-methoxy-leoligin led to a significant increase in capillary formation of microvascular endothelial cells (MVEC) on an artificial extracellular matrix (an exemplary matrix is available under the trade name Matrigel®). 5-methoxy-leoligin also significantly enhanced both the length and number of angiogenic sprouts of MVECs in a 3-dimensional sprouting assay. Furthermore, the pro-angiogenic activity of 5-methoxy-leoligin has been tested in vivo in the chorioallantois membrane (CAM) assay of chicken embryos. In the CAM assay, it is demonstrated that 5-methoxy-leoligin significantly increased the vascular density in vivo. These assays are recognized in the art to represent functional assays for in vitro and in vivo angiogenesis; see Methods in Endothelial cell biology, H G Augustin Ed.; 2004, Springer Lab Manual, ISBN 3-540-21397-X, Springer —Verlag Berlin, Heidelberg, N.Y..

This angiogenetic effect of compounds of formula (I), in particular of leoligin and more particular of 5-methoxy-leoligin and its derivatives was an unexpected finding. The prior art had disclosed an inhibition effect of leoligin on intimal hyperplasia of venous bypass grafts and it was shown that leoligin did not induce cell death, neither in small muscle cells nor in endothelial cells; see Reisinger et al. (2009), loc. cit. Furthermore, the prior art had documented that extracts and constituents of Edelweiss have enhancement properties in cholinergic transmission and show some memory improving properties; see Schwaiger et al. (2007), Planta Med 73, e-publication in Thieme eJournals or Hornick et al. (2008), Biochem Pharmacol. 76, 236-248. Yet, the use of leoligin and in particular the use of 5-methoxy-leoligin and its derivatives for stimulating angiogenesis or treatment of hypovascularity and/or the prevention and/or treatment of an angiogenic disorder has neither been described nor proposed in the art. Only the experimental data provided herein provide for the surprising finding that compounds of formula (I) and in particular leoligin and more particularly 5-methoxy-leoligin (and the derivatives of these compounds) have angiogenetic effects and can be used in the medical intervention of hypovascularity and/or of an angiogenic disorder. This surprising angiogenesis stimulation and/or promotion was not disclosed or proposed in the prior art.

Accordingly, it is evident from the disclosure herein and from the appended examples that the pro-angiogenic (angiogenesis stimulating/promoting) activity not only of 5-methoxy-leoligin but of compounds of formula (I) in general (like, inter alia, further (di)methoxy-derivatives of leoligin and also leoligin itself), can easily be demonstrated and measured by corresponding assays disclosed herein below and known in the art. As explained herein, it is easily conceivable that all compounds of formula (I) exert the pro-angiogenic effect and thus stimulate angiogenesis. Therefore, compounds of formula (I) are highly beneficial in a medical context where such a stimulation is desired. As detailed herein below, a skilled person has no problems in identifying subjects/patients where such a stimulation of angiogenesis is desired. For example, hypovascularity (i.e. a lack or undersupply in a certain tissue or organ with blood vessels) in a subject/patient may be one condition, where an increase or induction of angiogenesis is indicated. Accordingly, the treatment or prevention of diseases or disorders associated with hypovascularity is envisaged herein.

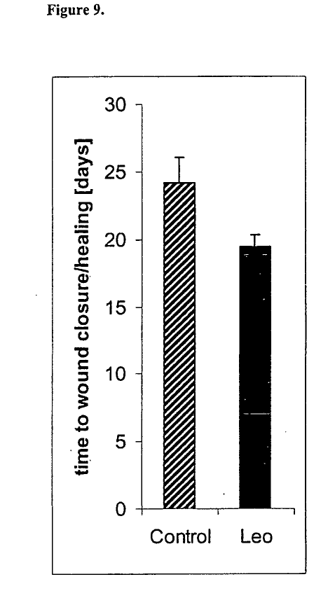

Thus, the herein described pharmaceutical composition comprising compounds of formula (I), like the preferred compound leologin—IUPAC name R2S,3R,4R)-4-(3,4-dimethoxybenzyl)-2-(3,4-dimethoxyphenyptetrahydrofuran-3-yl]methyl (2Z)-2-methylbut-2-enoat], or the even more particularly 5-methoxy-leoligin (IUPAC name: [(2S,3R,4R)-4-(3,4-dimethoxybenzyl)-2-(3,4,5-trimethoxyphenyptetrahydrofuran-3-yl]methyl-(2Z)-2-methylbut-2-enoat) and derivatives thereof can be employed in stimulating angiogenesis and/or preventing and/or treating angiogenic disorders and preventing or treating hypovascularity. Exemplary diseases and disorders wherein angiogenesis is required and desired (like, inter alia, non-diabetic retinopathy, microangiopathy, wound healing disorders, stable angina abdominalis, ischemic disorders or ischemic diseases, vascular dementia, impotence (penile dysfunction), etc.) or exemplary medical conditions (like, inter alia, wound healing, reactivation of necrotic tissue, revascularization for example after amputation or trauma/traumatic insult, etc.) are described herein below. As demonstrated in the appended Examples, the herein defined and described compounds of formula (I), such as the exemplary compound leoligin, can successfully be used to stimulate wound healing; accordingly, the compounds are useful in a medical setting where the stimulation of angiogenesis and/or the prevention and/or treatment of angiogenic disorders/diseases comprises revascularisation during or after wound healing as defined herein; see in particular Example 4 and FIG. 9.

However, the promotion or induction of angiogenesis may also be indicated in cases which do not necessarily involve hypovascularity, e.g. thrombosis, thromboembolism and the like.

One mechanism underlying angiogenesis is the formation of vessels by capillary sprouting and endothelial tube formation. The cells/cell type involved in this mechanism is primarily microvascular endothelial cells (MVECs). MVECs are, in general, physiologically a major cell type involved in the initiation of angiogenesis. The assays described herein and used in the appended examples reflect the major steps in angiogenesis (migration, sprouting, tube formation, capillary formation) and, are therefore true model systems of angiogenesis. It is believed that the herein provided compounds are particularly effective in stimulating the migration of MVECs (microvascular endothelial cells) and, thereby, in stimulating angiogenesis, at low concentrations as described and defined herein above and below.

Importantly, in vivo angiogenesis can directly be demonstrated in the chorioallantois membrane assay/CAM assay (see also below). Accordingly, these methods/assays demonstrate and prove the pro-angiogenic effect of 5-methoxy-leoligin (Lag2). Such assays can also be used to show the stimulation of angiogenesis by compounds of formula (I), such as other (di)methoxy-derivatives of leoligin and/or leoligin. The following documents also describe that MVECs are an excellent model system for assessing the stimulation of angiogenesis by certain compounds: Kern (2009), Blood 29;114(18):3960-7; Kern (2009), BMC Cancer. 17; 9:284. These technologies can be used to validate the angiogenesis potential of a given compound to be tested. Such technologies and assays may also be employed or used to validate further compounds subsumed under formula (I) as disclosed herein (in particular leoligin- or 5-methoxy-leoligin (IUPAC name: R2S,3R,4R)-4-(3,4-dimethoxybenzyl)-2-(3,4,5-trimethoxyphenyptetrahydrofuran-3-yl]methyl-(2Z)-2-methylbut-2-enoat)-derivatives) for their potence in angiogenesis and/or their usefulness in medical intervention of hypovascularity and/or angiogenic disorders.

The present invention solves the above identified technical problem since, as documented herein below and in the appended examples, it was surprisingly found that a lignan derived from the roots of Edelweiss (Leontopodium alpinum Cass.), in particular leoligin and derivatives thereof and more particularly 5-methoxy-leoligin (IUPAC name: [(2S,3R,4R)-4-(3,4-di methoxybenzyl)-2-(3,4,5-trimethoxyphenyl)tetrahydro furan-3-yl]methyl-(2Z)-2-methylbut-2-enoat) exhibit a highly beneficial effect on angiogenesis in a medical setting. 5-methoxy-leoligin is also denominated herein as “LaG2”.

Another, even more surprising finding was that not only 5-methoxy-leoligin but compounds of formula (I) in general and derivatives of leoligin and of 5-methoxy-leoligin as described herein promote or stimulate angiogenesis. Without being bound by theory, it is believed that the methoxy-groups may contribute to an increase in lipophilicity of the compounds to be used in accordance with the present invention, thus possibly enhancing and/or facilitating their cellular uptake. It is believed that this may be one reason why e.g. 5-methoxy-derivatives of leoligin may be used at comparable or lower concentrations than leoligin.

In the appended experimental section herein below it is shown that the compounds of the present invention comprised in a pharmaceutical composition can successfully be used as a stimulator of angiogenesis in a choridallantois membrane (CAM) assay. Such an assessment can also be carried out in murine or rat models and also larger animals/animal models. An exemplary protocol is provided in the experimental section herein below. In particular, an exemplary protocol using a hind limb ischemia rat or mouse animal model to assess the efficacy of the particular compound known under the trivial name “5-methoxy-leoligin” is given in the appended examples. The mentioned rat and mouse models are a preferred animal model of hypovascularity which can be used in context of the present invention. A person skilled in the art is readily in the position to adapt this protocol to compounds of formula (I), such as (a) (di)methoxy-derivative(s) of leoligin, (e.g. 5-methoxy-leoligin or 5,5-methoxy-leoligin) or leoligin per se. The protocol may also be adapted to other large animal models and it may then be assessed that compounds of formula (I) as described herein stimulate angiogenesis also in large animals in vivo, for example animals with the pathological condition hypovascularity. Results obtained in rats or mice can, to a large extent, be extrapolated to humans.

The following assays may also (alone or in combination or in combination with other assays known in the art) be employed to validate the potence of (a) given compound(s) (like derivatives of compounds of formula (I), such as ((di)methoxy-)derivative(s) of leoligin (e.g. 5-methoxy-leoligin) and/or leoligin-derivatives):

Myocardial Infarction Rat Model:

Ultrasound based quantification of the cardiac ejection fraction; MR-based assessment of the ejection fraction and cardiac dynamics; Morphometric analyses based on MR data, histochemistry (HC) and immunohistochemistry (IHC); Analysis of capillary density and angiogenesis by IHC e.g. by CD31 staining (endothelial marker); Assessment of the infarction area by HC (e.g. Masson-Trichrome-Goldner staining); Detection of necrotic and fibrotic tissue by HC and IHC.

Hind Limb Ischemia Model:

Infrared analyses of leg temperature as a marker for blood supply, in vivo (confocal) microscopy based analyses of capillary density in nude mice by using fluorescence labelled lectins; IHC and HC based analyses of capillary density and detection of necrotic and fibrotic tissue; Macroscopic analyses of leg movement.

The hind limb ischemia model is a standard model for assessing the new formation of blood vessels by angiogenesis. The model may be used to analyse drug effect on human diseases like thrombosis, thromboembolism or embolism.

Analysis of Capillary Density and Angiogenesis by IHC in Biological Samples e.g. by CD31(Endothelial Marker) Staining/Labelling.

CD31 (also known as PECAM-1 or platelet endothelial cell adhesion molecule) is a marker for endothelial cells. CD31 stainings and labellings (for example with anti-CD31 antibodies) are known in the art. Such an anti-CD31 staining/labelling can also be employed on biological samples, like biopsy samples, from subjects, like human subjects. The antigen CD31 is recognized, for example, by antibody clone JC70A available from Daco AJS (Glostrup, Denmark). Such a staining can be carried out on biological samples, for example before and after treatment with a compound to be tested for its angiogenesis potential. Such a biological sample may comprise a tissue sample of a test animal to be sacrificed. However, a biological sample may also comprise, inter alia, a tissue sample of a patient to be treated in accordance with this invention and the corresponding tests and assays may be carried out on a tissue sample/biopsy taken before and after the corresponding treatment.

Assessment of an infarction area in a biological sample or specimen by HC (e.g. Masson-Trichrome-Goldner staining); Detection of necrotic and fibrotic tissue in a biological sample by HC and IHC.staining/labelling of (blood) vessels in biological samples/samples or in biopsy tissue. Again, also in these assays and technologies, (tissue) samples from test animals can be employed. However, also biopsy samples of patients or subjects in need of medical intervention may be employed. Again, such samples/specimens may comprise samples/specimens/biopsies taken before and taken after medical intervention with the Edelweiss compounds provided and disclosed herein, in particular leogin or leogin derivatives, like ((di)methoxy)derivatives of leoligin (e.g. 5-methoxy-leoligin).

In context of this invention, it has been surprisingly found that compounds of formula (I) as described herein above, such as ((di)methoxy)derivatives of leoligin (e.g. 5-methoxy-leoligin) can successfully be used in a medical setting for the stimulation of angiogenesis. Compounds of formula (I) as described herein are, in particular, capable of stimulating angiogenesis at low concentrations, in particular at low molar concentrations. In contrast to compounds known in the art, the compounds of the present invention are non-toxic and can be used in a low concentrations of about 10 nM, 20 nM, 30 nM, 40 nM, 50 nM, 60 nM, 70 nM, 80 nM, 90 nM, more preferably of about 100 nM, 200 nM, 400 nM, 600 nM, 800 nM, 900 nM and up to 1000 nM (1 μM). As “low concentration” of the compounds of formula (I) and its derivatives, like, e.g. ((di)methoxy)derivatives of leoligin (e.g. 5-methoxy-leoligin), also concentrations higher than 1 μM, such as of about 1 μM to 200 μM, in particular 1 μM to 150 μM or 1 μM to 100 μM, may be considered. Accordingly, the herein provided compounds are preferably used in the stimulation of angiogenesis and/or treatment and/or prevention of hypovascularity and/or angiogenic disorders/diseases at low concentrations, preferably below 200 μM, 190 μM, 180 μM, 170 μM, 160 μM, more preferably below 150 μM, 140 μM, 130 μM, 120 μM or below 110 μM and, more preferably, below 100 μM or even at lower concentrations, for example below 95 μM, 90 μM, 85 μM, 80 μM, 75 μM, 70 μM, 65 μM, 60 μM, 55 μM or below 50 μM.

Accordingly, the compounds are preferably used at concentrations of between 100 nM to 300 μM, more preferably of between 1 μM to 200 μM, or even more preferably between 1 μM to 150 μM. Also preferred are concentrations of between 1 μM to 100 μM or 1 μM to 50 μM. For example, the compounds may be used at concentrations of 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM or 50 μM.

In particular, the above values/molar concentrations refer to the concentration of the compound(s) of formula (I) in the herein provided pharmaceutical composition, preferably for non-systemic administration (e.g. parenteral administration (such as injections) or topical administration and the like) as defined and described herein below. It is preferred that the compounds as defined herein are administered to the site where angiogenesis is to be stimulated e.g. by parenteral administration (such as injections) or by topical administration (such as ointments, drops etc. as defined below). Accordingly, local/direct (non-systemic) administration (e.g. parenteral, topical and the like) is particularly preferred in context of the present invention. Local administration as defined herein also ensures that the compounds of formula (I) are present at the site where angiogenesis is to be stimulated preferably in the same concentration as in the pharmaceutical composition, i.e. in the molar concentrations as defined herein above and below. Furthermore, such a type of administration has the advantage that stimulation of angiogenesis only occurs at the desired site. Exemplary situations and sites where angiogenesis is desired are described herein.

The (molar) concentrations may refer to only one compound If only one compound is present in the pharmaceutical composition; the values, may, however, also refer to one or more compounds, if one or more compounds are present in the pharmaceutical composition (i.e. the concentration may reflect the concentration of all compounds of formula (I) present in the composition).

Also envisaged herein is the use of the compounds provided herein at higher concentrations, i.e. higher than 200 μM, for example, about 210 μM, 220 μM, 230 μM, 240 μM, 250 μM, 260 μM, 270 μM, 280 μM, 290 μM or about 300 μM. Thus, the pharmaceutical composition of the present invention comprising these compounds as disclosed herein is particularly useful in the treatment or prevention of hypovascularity and/or the stimulation of angiogenesis and/or the prevention and/or treatment of an angiogenic disorder. The attending physician or veterinarian can readily deduce the amount of compounds to be taken or to be administered to a subject in need of angiogenesis stimulation and/or in need of medical intervention of hypovascularity and/or an angiogenic disorder. The subject to be treated in accordance with this invention may be a human subject but may also be (an) animal, like warm blooded animals, e.g. horses, dogs, cats, cattle, sheep, fowl and the like.

The use of lignans as disclosed herein and described under formula (I) and formula (I) derivatives, like, for example ((di)methoxy)derivatives of leoligin (e.g. 5-methoxy-leoligin) for stimulation of angiogenesis and, accordingly, also in the treatment or prevention of diseases associated with hypovascularity has neither been proposed nor disclosed in the art. To the contrary, an anti-angiogenic activity of some lignans was reported; see Bai, J Biol Chem 278(37), 35501-7 (2003) and Bergman Clin Cancer Res 13(3), 1061-7 (2007).

In one embodiment, a compound as defined herein above under under formula (I) and formula (I) derivatives, like, for example ((di)methoxy)derivatives of leoligin (e.g. 5-methoxy-leoligin) or the pharmaceutical composition comprising said compound is for stimulating angiogenesis and/or preventing or treating an angiogenic disorder. The term angiogenesis used in context of the present invention means the generation of new blood vessels, in particular small blood vessels (in particular with an inner diameter/diameter of the lumen of less than 5 mm), mainly by sprouting from existing blood vessels. “Vessel” refers to blood vessels, i.e. arteries and/or veins, in particular small blood vessels, like capillaries. The term “small blood vessels” as used herein refers to blood vessels with an inner diameter/diameter of the lumen of less than 5 mm. Blood vessels can easily be detected e.g. by CD31 staining. The term “stimulation” (used herein interchangeably with “promotion”) as used herein means the initiation of increase, or acceleration of the formation of new vessels (e.g. capillaries). As demonstrated in the appended examples, compounds of formula (I) and its derivates, such as 5-methoxy-leoligin, are capable of stimulating angiogenesis, i.e. they are, for example, capable of inducing the formation of capillaries (capillaries being blood vessels with a lumen for transportation of liquid), increasing the length and number of capillary sprouts and of increasing the vascular density in a tissue. In sum, the appended examples demonstrate that compounds of formula (I) are potent stimulators/agonists of angiogenesis. In other words, the herein provided compounds of formula (I) have a pronounced pro-angiogenic effect or activity. A skilled person is easily in the position to determine the effect of compounds of under formula (I) and formula (I) derivatives, like, for example ((di)methoxy)derivatives of leoligin (e.g. 5-methoxy-leoligin) on angiogenesis, for example by taking advantage of the herein described and used methods/assays as well as routine statistical analyses. For example, a certain compound is capable of stimulating angiogenesis if the compound is capable of increasing one of the above parameters in an in vitro and/or in vivo assays in a statistically significantly manner (compared to a control, e.g. cells/tissues not treated) with the compound). The herein described compounds of formula (I) promote, in particular, the formation and growth of small blood vessels (e.g. the growth of newly formed capillaries).

As explained in more detail below, hypovascularity may, for example, be caused by impaired angiogenesis or agenesia of blood vessels. Hypovascularity may also be genetically caused and/or caused by medicaments. In context of the present invention the term “agenesia” means that blood vessels are absent/non-existent in a certain tissue and/or organ.

Impaired angiogenesis and agenesia of blood vessels may be related to:
a) impairment of angiogenesis or agenesia of blood vessels caused by medicaments;
b) impairment of angiogenesis or agenesia of blood vessels caused by radiation;
c) impairment of angiogenesis or agenesia of blood vessels caused by environmental factors;
d) impairment of angiogenesis or agenesia of blood vessels caused by (a) disease(s) (also including infectious. diseases); and/or
e) undersupply of blood vessels/agenesia of blood vessels (e.g. genetically caused undersupply/agenesia)

Medicaments may also cause hypovascularity, e.g. by inducing vessel damage, reducing the number of vessels (e.g. by necrosis), or occluding vessels. Also radiation therapy may cause vessel damage, a reduction of the number of vessels (e.g. by necrosis), or occlusion of vessels.

Hypovascularity may also be caused by environmental factors or by (a) disease(s). For example, hypovascularity may be caused by/associated with microangiopathy, stable angina abdominalis, vascular induced/caused impotence (penile dysfunction), vascular dementia, or diabetes. Hypovascularity may also be associated with lung disorders. Thus, the treatment/prevention of lung disorders or diseases, like hypovascularity of chronic obstructive pulmonary disorder, COPD) or hypovascularity of lung parenchyma in accordance with the present invention is also envisaged.

Hypovascularity may also be genetically caused. Genetically caused hypovascularity may lead to vessel damage, a reduction of the number of vessels or an occlusion of vessels.

Furthermore, hypovascularity may also be caused by trauma, i.e. vascular injury/damage. For example, hypovascularity may occur in retinopathy due to blood vessel trauma. In this context, treatment of hypovascularity may be indicated with the compounds of formula (I), such as 5-methoxy-leoligin, for example, in retinopathy or after amputation by trauma and subsequently during reconstruction by transplantation of tissues and/or organs. The herein described compounds of formula (I), in particular 5-methoxy-leoligin, are, accordingly, beneficial in the reconstitution of blood vessels after traumatic damage.

Necrotisised tissue may have been caused by vessel trauma or injury. After removal of such necrotisised tissue by surgical intervention, lignan compounds of formula (I), e.g. 5-methoxy-leoligin, are also beneficial in the revascularisation and wound healing.

In accordance with the above, the pharmaceutical composition comprising the herein defined lignan compound of formula (I), like 5-methoxy-leoligin, is particularly useful in the treatment or prevention of diseases and/or disorders which are characterized by or associated with poor or impaired angiogenesis (e.g. angiogenic disorders and/or hypovascularity). Stimulation of angionesis is, therefore, in particular desired in impaired wound healing or wound healing disorders, in thrombosis, thromboembolism, and/or embolism. Thrombosis, thromboembolism, embolism may also be genetically caused. The promotion of angiogenesis is also beneficial in the reactivation of necrotising tissues, whereby the necrosis is not caused by vascular damage, but, for example, by thrombosis.

The present invention relates in one embodiment to a pharmaceutical composition comprising a compound as defined herein for use in treating or preventing hypovascularity and/or treating or preventing angiogenic disorders. Also envisaged herein is the use of the herein described compounds of formula (I) for the preparation of a pharmaceutical composition for stimulating angiogenesis and/or for treating or preventing hypovascularity and/or treating or preventing angiogenic disorders. The term “hypovascularity” means in context of the present invention a lack or undersupply of blood vessels (in particular in (a) certain tissue(s) and/or organ(s)) in a subject compared to a healthy subject or to healthy tissue(s) and/or organ(s) in the same subject. A skilled person is easily in the position to assess whether (a) tissue(s) and/or (a) organ(s) is characterized by hypovascularity. An artisan is also easily capable of identifying appropriate controls for such an assessment. Hypovascularity will, for example, be diagnosed/assessed if the number, density or volume of blood vessels is (preferably statistically significantly) lower than in the control. Such a lack of blood vessels often results in an insufficient supply of the respective tissue(s) and/or organ(s) with e.g. nutrients and oxygen and may have further deleterious effects. In other words, hypovascularity can also be defined as an undersupply of (a) tissue(s) and/or organ(s) with blood vessels. Accordingly, hypovascularity may also be diagnosed/assessed based, for example, on the metabolic profile of a sample obtained from a subject (e.g. a biopsy sample). Hypovascularity may also be genetically inherited. Accordingly, also the treatment/prevention of hereditary diseases and disorders such as hereditary hypovascularity is envisaged in context of the present invention. The use of the herein described pharmaceutical composition is also indicated in cases where increased angiogenesis is beneficial, even though a subject does not suffer from (or is being prone to suffer from) poor or impaired angiogenesis (which may be the cause for hypovascularity).

A skilled person is easily in the position to identify specific pathological conditions, where stimulation of angiogenesis is indicated, e.g. angiogenic diseases or disorders. A skilled person is also aware of diseases and disorders associated with hypovascularity. As explained above, often the stimulation of angiogenesis will be particularly beneficial in context of hypovascularity. Exemplary pathological conditions/diseases/disorders to be treated in context of the invention are also described herein above and below.

In one particular embodiment, it is envisaged that the herein above described stimulation of angiogenesis and/or the treatment or prevention of hypovascularity and/or treatment or prevention of (an) angiogenic disorder(s) with the lignan compounds of formula (I), such as 5-methoxy-leoligin, comprises the treatment or prevention of one or more of the diseases/disorders described herein below. For example, the treatment of wound healing, in particular of traumatic wounds like surface and skin wounds is envisaged. The use of the lignan compounds of formula (I) is also indicated in wound healing in the recovery phase after surgical removal of necrotic tissue.

Lignan compounds of formula (I), such as 5-methoxy-leoligin can also beneficially employed in the treatment or prevention of retinopathy (non-hypertrophic, in particular non-diabetic) or vascular obliteration, wherein the latter preferably cannot be treated by surgery. Compounds of formula (I) also promote angiogenesis after amputation or after transplantation of (a) tissue(s) and/or (a) organ(s). Their use is therefore also indicated in such a context. Furthermore, lignan compounds of formula (I) can be used in the treatment or prevention of arterio- and vaso-microvasculopathy of blood vessels and/or arterio- and vaso-microangiopathy of blood vessels. The compounds are particularly useful when the microvasculopathy or microangiopathy cannot be treated by surgery. Also envisaged herein is the treatment of necrosis with lignan compounds of formula (I). For example, the herein described compounds are particularly useful in situations where the necrosis cannot be treated by surgery. The lignan compounds may then be helpful in reactivating (thereby revascularising) necrotisised tissue or hibernating tissue. Also envisaged herein is the use of lignan compounds of formula (I), in particular 5-methoxy-leoligin in the treatment or prevention of stable angina abdominalis, vascular dementia, impotence (i.e. penile dysfunction) and/or ischemic diseases and disorders (such as myocardial infarction and stroke) and/or non-cardial ischemic diseases or disorders. Again, the compounds of formula (I) are particularly advantageous in treating/preventing ischemic diseases, where surgical treatment is not possible.

In one embodiment, the present invention relates to a pharmaceutical composition comprising a compound of formula (I), in particular (di)methoxy-derivatives of leoligin (for example 5-methoxy-leoligin) and/or leoligin, for use in treating or preventing the herein described diseases and disorders. Also envisaged herein is the use of the herein described compounds of formula (I) for the preparation of a pharmaceutical composition for treating or preventing these disorders. The definitions and explanations regarding such diseases/disorders given herein apply mutatis mutandis in this context.

Preferably, the vascular obliteration is thrombosis, thromboembolism or embolism. In particular, thrombosis, thromboembolism or embolism which are not amenable to treatment by surgery (i.e. preferably where treatment by surgery is not possible), can be treated or prevented by the pharmaceutical composition comprising the lignan compound of formula (I), such as 5-methoxy-leoligin. The embolism may, inter alia, be chronic pulmonary embolism. Chronic pulmonary embolism is one example for an embolism that cannot be treated by surgery.

In particular, subjects suffering from the above defined vascular obliteration and/or hypovascularity and/or impaired/poor angiogenesis which preferably cannot be treated by surgery (because surgery is impossible e.g. in high risk patients) benefit from the above treatment or prevention according to the invention. The group of patients that cannot be treated by surgery is therefore preferably treated in accordance with the present invention. The vascular obliteration may occur in certain subjects in particular in small blood vessels, e.g. in capillaries and may, in addition, occur rather often. Also in such a case, treatment by surgery is not possible.

In a preferred embodiment of the present invention, wound healing and, in particular, wound healing disorders are to be treated. The term “wound-healing disorder” as used herein refers to a delayed or impeded healing of a wound. A wound healing disorder may be caused by any influences that delay or endanger the healing of a wound or lead to subsequent complications. Non-limiting reasons for wound healing disorders are wound hypoxia, infection, presence of debris and necrotic tissue, use of anti-inflammatory medications, a diet deficient in vitamins or minerals or general nutritional deficiencies, tumors, environmental factors, and metabolic disorders, such as diabetes mellitus. Lignan compounds of formula (I), such as 5-methoxy-leoligin, may be used to counteract the following causes of improper wound healing: wound hypoxia, presence of debris and necrotic tissue, a lack of wound tissue supply with vitamins or minerals or other nutrients (the supply of wound tissue by new blood vessels will improve tissue availability of those factors). Also metabolic disorders, such as diabetes mellitus in which altered angiogenesis and/or the lack of functional vessels may be associated with wound healing disorders, i.e. a subject suffering from e.g. diabetes will also often experience a worse wound healing than a healthy individual. In particular (a) traumatic wound(s) are to be treated and also the treatment of surface and skin wounds is envisaged herein. Wounds can also be treated in the recovery phase after surgery, for example, in order to accelerate the healing process.

As demonstrated in the appended examples, compounds of formula (I), in particular (di)methoxy-derivatives of leoligin (for example 5-methoxy-leoligin) and/or leoligin, promote the migration of endothelial cells in damaged areas and thus stimulate wound healing. Particularly preferred is the use of the herein described pharmaceutical composition comprising lignan compounds of formula (I) in wound healing upon amputation and/or transplantation, in particular transplantation of (a) tissue(s) and/or (an) organ(s). In such a context it is also envisaged that wound healing is to be stimulated in damaged or injured blood vessels. As explained above, transplantation of blood vessels is not envisaged in this context. For example, after amputation (and, optionally, after reattaching an amputated body extremity (reconstruction by surgery after amputation), e.g. if the body extremity has been removed by trauma) promotion of angiogenesis is indicated.

As mentioned above, also arterio- and veno-microangiopathy that preferably cannot be treated by surgery is to be treated or prevented in accordance with the present invention. This treatment or prevention of arterio- and veno-microangiopathy may also comprise the treatment of diabetes. Accordingly, also diabetic arterio- and veno-microangiopathy, diabetic nephropathy and/or diabetic neuropathy can be treated/prevented.

Preferably, microangiopathy is microangiopathy of the retina, of the brain, or of the ear. Also the treatment or prevention of thrombotic arterio- and veno-microangiopathy is envisaged herein and the treatment of arterio- and veno-microangiopathic haemolytic anemia is also envisaged. Also envisaged herein is the treatment of blood vessel injuries or damages, in particular traumatic blood vessel injuries or damages. The herein provided and described pharmaceutical composition is also useful in the reactivation of necrotisising tissue or reactivation of hibernating tissue, in particular when surgery is not possible or where conservative therapy is indicated. For example, a post ischemic scar or hibernating myocardium upon myocardial infarction (i.e. in the recovery phase) may be reactivated and thus treated. It is of note that the patients to be treated in this context have already experienced a myocardial infarction, whereas, for example, patients suffering from coronary artery disease (CAD) have not (yet) experienced a myocardial infarction. Myocardial infarction (MI) or acute myocardial infarction (AMI) is also commonly known as a heart attack. The patients to be treated in accordance with the present invention are in the recovery phase after myocardial infarction.

In particular, the pharmaceutical composition comprising compounds of formula (I), in particular 5-methoxy-leoligin, is beneficial in context of a conservative therapy and/or therapy in support of conventional therapies of the above described diseases and disorders. The terms “conservative therapy” and “therapy in support of conventional therapies” are well known in the art.

“Conservative therapy” is known as patient care management of a clinical condition with the least aggressive of available therapeutic options and refers in context of the present invention in particular to “non-invasive therapy”. This term reflects the fact that e.g. no therapy by surgery or other invasive therapies are applied. Though, for example, injections may be considered as invasive, they are usually not regarded as “invasive therapy”. Conservative therapy is, for example, envisaged in ischemia/ischemic diseases or disorders such as post ischemic scars upon myocardial infarction. Thus, the patient group subjected to conservative therapy and/or therapy in support of conventional therapies is preferred in context of this invention. In particular a patient/patient group subject to conservative therapy is preferred herein. Particularly envisaged is conservative therapy of angina abdominalis (the latter comprising ((non-fulminant) mesenteric infarction).

Therapy in support of conventional therapies refers to any form of treatment intended to relieve symptoms or help the patient live with them rather than attempt changes in character. Accordingly, also “supportive therapy” in the context of palliative medicine (palliative therapy) is envisaged in context of the present invention, e.g. in patients where treatment by surgery is not possible, for example if the patient is polymorbid or final.

As mentioned above, also vascularly caused/induced impotence (penile dysfunction) may be treated or prevented in accordance with the present invention.

Subject/patients to be treated in accordance with the present invention are, in particular, patients having vascular problems and/or suffering from (or being prone to suffering from) infarctions and/or ischemias. These patients can, in particular, not be treated by surgery or percutaneous coronary intervention (PCI). Reasons for exclusion from treatment by surgery or PCI are, for example, bad general condition, site not amenable to surgery (e.g. retina or (an) other tissue(s)/organ(s) with small blood vessels/capillaries).

Prevention of the herein above defined diseases/disorders (e.g. prevention of stable angina abdominalis) is indicated in accordance with the present invention in subjects/patients which have not (yet) been treated by surgery or PCI. Also patients having systemic problems (such as patients suffering from diabetes) are to be treated, in particular in order to prevent the herein described disorders and diseases. In particular, in this context, patients with microvascular problems (e.g. arterio- and veno-microangiopathy) benefit from a treatment according to the invention. As pointed out above, in preferred and illustrative embodiments of the present invention compounds of formula (I) and formula (I) derivatives, like, for example ((di)methoxy)derivatives of leoligin (e.g. 5-methoxy-leoligin) can be used in the medical or pharmaceutical intervention of angiogenesis disorders/diseases, wherein stimulation of angiogenesis is desired. These compounds are also useful, as described herein, in the treatment or prevention of hypovascularity and corresponding disorders. Situations and disorders wherein the stimulation of angiogenesis is desired comprise, but are not limited to, revascularisation during or after wound healing and/or during scar tissue formation on skin or non-cardiac inner organs, like glomerular scarring/glomerulosclerosis, revascularisation after or during amputation, trauma, surgery and/or transplantation of tissues or organs and/or the (re-)activation of vessel growth after or during necrosis. The treatment or prevention of hypovascularity with said compounds of formula (I) and its derivatives comprises, inter alia and not limiting, the treatment or prevention of a disease/disorder selected from the group consisting of wound healing disorder, vascular obliteration, arterio- and veno-microvasculopathy, arterio- and veno-microangiopathy, ischemic diseases or disorders, non-cardial ischemic diseases or disorders, disorders relating to hibernating tissue, stable angina abdominalis, vascular dementia, and impotence or penile dysfunction.

Also combination therapy of the herein described pharmaceutical compositions comprising compounds of formula (I), in particular 5-methoxy-leoligin is envisaged herein, e.g. in combination with VEGF therapy.

In one embodiment, the present invention relates to a method for stimulating angiogenesis comprising the administration of a compound of formula (I) as defined herein above to a subject in need thereof. Furthermore, one embodiment of the present invention relates to the treatment or prevention of hypovascularity in a subject suffering from or being prone to suffering therefrom, wherein a compound as defined herein is to be administered to said subject. The definitions and explanations given herein in respect of the treatment of particular diseases and disorders applies here, mutatis mutandis. It is preferred that the subject is a human, however also animals, as pointed out above, may be treated by the means and methods provided herein.

The herein above described disorders and diseases are also provided in a standardized form by the World Health Organisation (WHO) and the corresponding definitions can be retrieved from the WHO ICD-10 Version 2007 (available online via http://apps.who.int/classifications/apps/icd/icd10online/) The following table provides a non-exhaustive list of certain diseases and disorders to be treated or prevented in accordance with the invention or certain medical conditions wherein the use of compounds of formula (I) and its derivatives, like leoligin, 5-methoxy-leoligin, etc. may be employed. The accession numbers (referring to WHO ICD-10; Version 2007) for such diseases, conditions and disorders are also provided in the following, non-limiting table:

| | |
|---|---|
| wound healing/wound healing disorders | T79.9 |
| Retinopathy and maculopathy caused by | H34, -; H36.8 |
| hypovascularity | |
| Post traumatic (i.e. damaged and injured) | T14.5 |
| blood vessels | |
| Frostbite | T33-T35; T69.0, .1, .8, |
| | .9; |
| microarteriopathy and microvenopathy | I73* E13.5-; E13.74; |
| | E13.75; |
| necrotisising tissue e.g. in gangrene; | R02; T33-T35; J85-J86; |
| frostbite, abscess as a result of vascular | T87.5; J38.7; |
| obliteration and hypovascularity | |
| Ischemic tissue as a result of vascular | N28.0 H34.-G45.- |
| obliteration or hypovascularity | |
| thrombosis, thromboembolism, embolism of | I82.-; I74.-; I80.-; I81; |
| small arteries or veins | G08; I26.-; I65.-; I66.-; |
| | I82.9; I82.8 |
| post infarction, e.g. of the kidneys | N.28 |
| amputation by trauma and reconstruction by | S18; S58.-; S08.-; S88.-; |
| surgery | S68.-; S48.; S78.-; S98.-; |
| | T87.-; S28.-; |
| Lack or undersupply of blood vessels/agenesia | Q27, -; Q28.-; I99 |
| e.g. in congenital absence of blood vessels in | |
| parts of the body | |
| angina abdominalis | K55.- |
| e.g. non-fulminant mesenteric infarction | |
| vascular dementia | F01, -; |
| vascular induced/caused impotence (penile | N84.4 or N48.4 |
| dysfunction) | |

The terms “treatment”, “treating” and the like are used herein to generally mean obtaining a desired pharmacological and/or physiological effect. The effect may be prophylactic in terms of completely or partially preventing a disease or symptom thereof (referred to herein as “prevention” or “preventing”) and/or may be therapeutic in terms of partially or completely curing a disease and/or adverse effect attributed to the disease (referred to herein strictu sensu as “treatment” or “treating”). In particular, the term “prevention” as used herein refers to preventing a disease in a subject which may be predisposed to the disease. Though the term “treatment” as used herein covers any treatment of a disease in a subject, “treatment” refers in particular to inhibiting the disease, i.e. arresting its development; or relieving the disease, i.e. causing regression of the disease.

A “patient” or “subject” for the purposes of the present invention includes both, humans and other animals, particularly warm blooded animals and/or mammals, and other organisms. Thus, the methods are applicable to both human therapy and veterinary applications. In the preferred embodiment the patient is a mammal, and in the most preferred embodiment the patient is human.

As already mentioned and explained above, it is particularly preferred that said subject/patients to be treated in accordance with the present invention, are preferably treated by conservative therapy, i.e. by non-invasive therapy, like parenteral or oral administration (like, e.g. intraveneous, intraarterial, subcutaneous, intraosseous, intramuscular or intradermal administration) or topical administration (like (sub-)cutaneous, epicutaneous, by inhalation, i.e. transdermal, transmucosal) of the compounds of formula (I) and its derivatives. Accordingly, preferred routes of administration are the parenteral route, oral route, intravenous route, subcutaneous route, intranasal route or transdermal route. As explained above, it is preferred that the pharmaceutical composition of the present invention is directly (non-systemically) administered to the site where angiogenesis is to be stimulated. Also preferred herein is the treatment of subjects/patients that are in recovery after surgery (i.e. in these cases where a certain disease can be or was treated by surgery), e.g. patients where a post-ischemic scar upon myocardial infarction is to be reactivated and thereby treated. The treatment of this patient group and/or stimulation of angiogenesis in this patient group is also particularly preferred herein.

As mentioned above and shown in detail in the experimental section herein below, the compound to be used in accordance with the present invention or the compound as comprised in the pharmaceutical composition of the present invention may be obtained from plants belonging to the genus Leontopodium, optionally followed by standard derivatization reactions. It is particularly preferred that the compounds provided herein may be obtained from Leontopodium alpinum, in particular Leontopodium alpinum Cass., which is commonly known under the trivial name “edelweiss”. According to another nomenclature “edelweiss” may also be known under the scientific term “Leontopodium nivale subsp. alpinum (Cass.) Greuter”. However, the terms “Leontopodium alpinum Cass” and “Leontopodium nivale subsp. alpinum (Cass.) Greuter” refer to the same plant species and merely reflect a regrouping of the species in botanical nomenclature. Accordingly, these terms can be used interchangeably in context of the present invention and any definitions and explanations given herein in respect of Leontopodium alpinum Cass. also apply to Leontopodium nivale subsp. alpinum (Cass.) Greuter, mutatis mutandis, and vice versa.

It is envisaged herein that the compounds to be used according to the present invention may be obtained from other Leontopodium species, including but not limited to commercial cultivars, such as Leontopodium hybrids. Accordingly, the compounds may be obtained from the following, exemplary Leontopodium species and cultivars: L. catipes (DC.) F. Muell., L. gnaphalioides Hieron., L. japonicum var. sandwicense H. Lév., L. linearifolium Britton, L. meredithae (F. Muell.) F. Muell., L. albogriseum Hand.-Mazz., L. aloysiodorum Hort. ex Hand.-Mazz., L. alpinum Cass., L. alpinum Colm. ex Willk. & Lange, L. alpinum Cass. subsp. nivale (Ten.) Tutin, L. amrheinii Hort. ex Mollers, L. andersonii C. B. Clarke, L. antennarioides Socz., L. arbuscula Beauverd, L. artemisiifolium Beauverd, L. aurantiacum Hand.-Mazz., L. beerianum Beauverd ex Murr, L. blagoveshczenskyi Vorosch., L. bonatii Beauverd, L. brachyactis Gand., L. caespitosum Beauverd, L. caespitosum Diels, L. calocephalum Beauverd, L. campestre Hand.-Mazz., L. catipes F. Muell., L. chamaejasme Beauverd, L. charkeviczii V. Yu. Barkalov, L. chuii Hand.-Mazz., L. conglobatum Hand.-Mazz., L. coreanum Nakai, L. dedekensi Beauverd, L. delavayanum Hand.-Mazz., L. discolor Beauverd, L. dubium Beauverd, L. evax Beauverd, L. fangingense Ling, L. fauriei Hand.-Mazz., L. fedtschenkoanum Beauverd, L. fimbrilligerum J. R. Drumm., L. fischerianum Beauverd, L. foliosum Beauverd, L. forrestianum Hand.-Mazz., L. francheti Beauverd, L. futtereri Diels, L. giraldii Diels, L. gnaphalioides Hieron. ex Sod., L. gracile Hand.-Mazz., L. haastioides Hand.-Mazz., L. hallaisanense Hand.-Mazz., L. haplophylloides Hand.-Mazz., L. hastatum Beavera, L. hayachinense (Takeda) Hara & Kitam., L. helveticum D. Don ex G. Don, L. himalayanum DC., Leontopodium×intermedium Sunderm., L. jacotianum Beauverd, L. jacotianum Beauverd var. haastioides (Hand.-Mazz.) R. C. Srivastava, L. jamesonii Beauverd, L. japonicum Miq., L. japonicum Miq. L. happoense Hid.Takah. ex T. Shimizu, L. javanicum Zoll. & Mor., L. junpeianum Kitam., L. kamtschaticum Komarov, L. krasense Derganc, L. kurilense Takeda, L. leiolepis Nakai, L. leiolepis Nakai var. crinulosum H. S. Pak, L. leiolepis Nakai var. curvicollum H. S. Pak, L. leontopodinum Hand.-Mazz., L. leontopodioides Beauverd, L. leontopodium Karst., Leontopodium×lindavicum Sunderm., L. linearifolium Britton, L. linearifolium Benth. & Hook. f., L. linearifolium Hand.-Mazz., L. longifolium Ling, Leontopodium×macranthum Sunderm., L. maireanum Beauverd ex Hand.-Mazz., L. makianum Kitam., L. mariae Muell., L. melanolepis Ling, L. meredithae F. Muell., L. micranthum Ling, L. microcephalum (Hand.-Mazz.) Ling, L. microphyllum Hayata, L. monocephalum Edgew., L. monoicum Benth. & Hook. f., L. montisganeshii S. Akiyama, L. muscoides Hand.-Mazz., L. nanum Hand.-Mazz., L. nivale (Ten.) Huet ex Hand.-Mazz., L. nivale (Ten.) Huet ex Hand.-Mazz. subsp. alpinum (Cass.) Greuter, L. niveum Hand.-Mazz., L. nobile Beauverd, L. ochroleucum Beauverd, L. ochroleucum Beauverd subsp. campestre (Ledeb.) V. M. Khanminchun, L. ochroleucum Beauverd subsp. campestre (Hand.-Mazz.) Khanm., L. ochroleucum Beauverd subsp. conglobatum (Turcz.) V. M. Khanminchun, L. ochroleucum Beauverd subsp. conglobatum (Hand.-Mazz.) Khanm., L. omeiense Ling, L. palibinianum Beauverd, L. paradoxum J. R. Drumm., L. perniveum Honda, L. pirinicum Hand.-Mazz., L. pulchellum Beauverd, L. pusillum Hand.-Mazz., L. roseum Hand.-Mazz., L. rosmarinoides Hand.-Mazz., L. sachalinense Miyabe & Kudo, L. sandwicense Rock, L. shinanense Kitam., L. sibiricum Cass., L. sinense Hemsl. ex Forb. & Hemsl., L. smithianum Hand.-Mazz., L. souliei Beauverd, L. spathulatum Kitam., L. stellatum A. P. Khokhr., L. stoechas Hand.-Mazz., L. stoloniferum Hand.-Mazz., L. stracheyi C. B. Clarke ex Hemsl., L. subulatum Beauverd, L. suffruticosum Y. L. Chen, L. tataricum Kom., L. thomsonianum Beauverd, L. umbellatum Bluff. & Fingerh., L. villosulum A. P. Khokhr., L. villosum Hand.-Mazz., and L. wilsonii Beauverd.

The compounds provided herein may also be obtained from corresponding cell culture, cell suspension culture or a comparable in vitro cultivation technique, such as callus culture and the like. A person skilled in the art will be aware of corresponding means and methods for establishing and maintaining corresponding cultures. In a preferred embodiment of the invention, the cell culture is derived from roots of Leontopodium species described herein above, in particular Leontopodium alpinum (edelweiss). Most preferably, the cell culture is derived from hairy roots.

Based on his general knowledge and the teaching provided herein a skilled person is readily in the position to obtain the compounds to be used herein, in particular 5-methoxy-leoligin and/or leoligin (and (di)methoxy-derivatives thereof), from Leontopodium species. Generally, the person skilled in the art is capable of preparing an extract from plants belonging to the genus Leontopodium by standard techniques. A preferred method for extracting these compounds from the roots of Leontopodium alpinum is provided in Example 1 herein below. An artisan will be aware how to adapt this protocol for extracting the compounds from further Leontopodium species and in particular from roots of these plants. A skilled person will also be aware of alternative protocols to be used in this context. The term extract is well known in the art and used accordingly herein. For example, this term may refer to preparations of fluid consistence (fluid extracts and tinctures), semisolid consistence (viscous extracts, syrup concentrate) or solid consistence (dried extracts), which are usually prepared using fresh or dried plant material.

The extract obtained from Leontopodium species is an extract that is received by the use of an organic or non-organic solvent. Suitable solvents are hexane, heptane, petroleum benzene, acetone, chloroform, dichloromethane, ethyl acetate, diethylether, liquid carbon dioxide, ethanol, ternary butyl methyl ether (tBMe) and mixtures of water and alcohol. The extract may be obtained by extracting the plant material, in particular roots, with any of the solvents separately. It is further possible to subsequently extract the obtained extract with a second solvent or mixtures of different solvents. An exemplary, non-limiting solvent to be Used in a first extraction step is hexane. However, any of the above solvents can be used in such a first extraction step. This first extraction step may be followed by (a) subsequent second (or further) extraction step with at least one of the above exemplary solvents, e.g. dichloromethane, chloroform or ternary butyl methyl ether (tBMe). Extraction of the compounds disclosed herein (in particular compounds of formula (I), such as (di)methoxy-derivative(s)) of leoligin (e.g. 5-methoxy-leoligin) and/or leoligin) in accordance with the present invention is also illustrated in the appended examples. Preferably, dichloromethane and methanol are used as extraction solvents. In subsequent extraction, it is preferred that the compounds are first extracted with n-hexane, followed by a subsequent extraction with dichloromethane, chloroform or tBMe. As shown herein, the lignan content (i.e. content of compounds of formula (I), such as (di)methoxy-derivative(s)) of leoligin (e.g. 5-methoxy-leoligin) and/or leoligin) can be increased by a second or further extracting steps using the herein described methods, and in particular the above solvents. Also the use of chromatographic methods, such as Sephadex-LH20-column chromatography and in particular silica gel column chromatograph is advantageous in this context. As also demonstrated in the appended examples, an increase in the leoligin content from about 0.7% to about 2.2% can be achieved using Sephadex-LH20-column chromatography. It is shown herein that a pronounced increase in the leoligin content from about 1.4% to about 10% can be achieved using silica gel chromatography.

It is envisaged herein that further chromatographic methods to increase the content of the herein disclosed compounds (in particular compounds of formula (I), such as (di)methoxy-derivative(s)) of leoligin (e.g. 5-methoxy-leoligin) and/or leoligin) can be used in addition or in the alternative to the above described methods. Exemplary, non-limiting chromatographic methods to be used in this context are reversed phase column chromatography or (semi)-preparative HPLC using water/acetonitrile mixtures or comparable solvent mixtures known in the art. Alternatively, techniques of liquid-liquid extractions (discontinuous or continuous methods) can be used to increase the content of the herein disclosed compounds (in particular compounds of formula (I), such as (di)methoxy-derivative(s)) of leoligin (e.g. 5-methoxy-leoligin) and/or leoligin). An exemplary liquid-liquid extraction is high speed counter current chromatography using a solvent system of two not mixable solvents.

The preparation of the basic extract of Leontopodium species, in particular Leontopodium alpinum, may comprise mechanical pulping, sonication, use of mortars and pestles, freeze-thawing cycles, use of blenders (like Waring-Blenders, Polytron), liquid homogenization and maceration (see also appended examples), or e.g. Dounce homogenization, Potter-Elvehjem, French Press etc. In the appended examples, a mechanical maceration is used. However, the extracts may be obtained by disrupting the cells and cells from the Leontopodium species by any mechanical/physical or chemical means, like by use of detergents.

Mechanical methods rely on the use of rotating blades to grind and disperse large amounts of complex tissue, such as plant leaves, flowers, seeds, and in particular roots. The Waring blender and the Polytron are commonly used for this purpose. Unlike the Waring blender, which is similar to a standard household blender, the Polytron draws tissue into a long shaft containing rotating blades.

Liquid-based homogenization is the most widely used cell disruption technique for cultured cells. Cells are lyzed by forcing the cell or tissue suspension through a narrow space, thereby shearing the cell membranes. Three different types of homogenizers are in common use. A Dounce homogenizer consists of a round glass pestle that is manually driven into a glass tube. A Potter-Elvehjem homogenizer consists of a manually or mechanically driven Teflon pestle shaped to fit a rounded or conical vessel. The number of strokes and the speed at which the strokes are administered influences the effectiveness of Dounce and Potter-Elvehjem homogenization methods. Both homogenizers can be obtained in a variety of sizes to accommodate a range of volumes. A French press consists of a piston that is used to apply high pressure to a sample volume of 40 to 250 ml, forcing it through a tiny hole in the press. Only two passes are required for efficient lysis due to the high pressures used with this process. It is of note that in more industrial applications also other, larger devices may be employed to prepare the extracts from Leontopodium species.

Sonication is also a physical disruption commonly used to break open cells. The method uses pulsed, high frequency sound waves to agitate and lyse cells and finely diced tissue. To prevent excessive heating, ultrasonic treatment may be applied in multiple short bursts to a sample immersed in an ice bath. Sonication is best suited for volumes <100 ml.

The freeze/thaw method is commonly used to lyse bacterial and cells from higher organism. The technique involves freezing a cell suspension in a dry ice/ethanol bath or freezer and then thawing the material at room temperature or 37° C. This method of lysis causes cells to swell and ultimately break as ice crystals form during the freezing process and then contract during thawing. Multiple cycles are necessary for efficient lysis, and the process can be quite lengthy.

Cells, organisms as well as tissue might be treated with various agents to aid the disruption process. Chemical substances, such as hexane, petroleum benzene, chloroform, dichloromethane, acetone, ethyl acetate, diethyl ether, ethanol and mixtures of water and alcohol or mixtures of different solvents may be added during or before mechanical disruption. Lysis can also be promoted by suspending cells in a hypotonic buffer, which causes them to swell and burst more readily under physical shearing. Processing can be expedited by treating cells with glass beads in order to facilitate the crushing of cell walls. Viscosity of a sample typically increases during lysis due to the release of nucleic acid material. DNase may be added to samples along with to reduce this problem.

Less preferred, however envisaged, is the use of detergents in the preparation of the extracts to be treated in accordance with the present invention. Detergents are a class of molecules whose unique properties enable manipulation (disruption or formation) of hydrophobic-hydrophilic interactions among molecules in biological samples. Such detergents may be used to lyse cells, solubilize membrane proteins and lipids. Generally, moderate concentrations of mild (i.e., nonionic) detergents compromise the integrity of cell membranes, thereby facilitating lysis of cells and extraction of soluble protein, often in native form. Using other conditions, detergents effectively penetrate between the membrane bilayers at concentrations sufficient to form mixed micelles with isolated phospholipids. Detergents may be, e.g. Triton X-100®, Triton-X-114®, NP-40®; CHAPS, Tween-20®, Tween-40®, Tween-80®, Octyl Glucoside, Octylthio Glucoside, Brij-35, Brij-58, SDS and the like. However, it may be useful to stabilize the extract by certain chemical means. Illustrative stabilizers are discussed herein below in context of pharmaceutical or cosmetic compositions.

The cells and plants to be employed in order to obtain the basic extract may be cells of natural origin as well as cultured cells or plants. It is preferred herein that the cells or plants and in particular roots of the plants are dried before mechanical disruption/maceration as described herein above. The cells or plants may be air dried, lyophilized (freeze-dried) or, though less preferred, dried in an oven. It is preferred herein that the “cell(s)” and “plant(s)” to be used as a basic material are fresh, i.e. harvested shortly before the extract is prepared. Nonetheless, it is possible to store the basic material before its use in the preparation of the extract. For example, the basic material may be lyophilized (freeze-dried) or simply frozen. and stored at low temperatures, e.g. at about −20 to −30° C. or as low as −80° C.

In context of the present invention, the term “cell” and “plant” to be used as basic material for preparing the extract to be treated by the method of the present invention also comprises the use of “tissues”. Such tissues may be leaves, sprouts, or reproductive organs e.g. flowers. Preferably, the tissues are roots, in particular hairy roots. In addition, callus or cell cultures may be used which may be derived from tissues described above, in particular roots, and which are grown in liquid culture or on solidified culture medium. The appropriate culturing methods of calli or cell cultures are known to a person skilled in the art. A culture medium may be for example a MS (Murashige and Skoog) medium while a solidifying agent may be agarose, plant agar or bacto agar. A basic culture medium such as a MS medium may be modified in respect to pH range, carbon or nitrogen source, amino acids or vitamins amongst others. The use of plants regenerated from such callus or cell culture is also envisaged, as well as plants or organisms generally grown or propagated in vitro.

Methods for preparing the extract are known in the art and also described herein. Preferably, the extract is further processed shortly after its preparation (e.g. the extract is used in the preparation of a herein disclosed pharmaceutical composition); however, it is also possible to store the extract for some time before they are used in accordance with the present invention.

The extracts may, for example, be stored in lyophilized form or in form of dried extracts. However, each storage form known in the art is be employed, as long as the storage has the effect that the extract (and its components) remain efficacious over a long time period, i.e. the stored extract has, preferably, substantially the same efficacy as the fresh extract.

Dried extracts can be routinely prepared by methods known in the art. For example, following mechanical disruption of the basic (plant) material by e.g. maceration or percolation, the material can be extracted using (a) solvent(s) or mixtures thereof as described herein. After separation of the fluid phase and the extract residue (which contains e.g. cellulose, pectin and the like and which does, preferably, not contain the active substance(s) as disclosed herein, i.e. predominantly compounds of formula (I), such as (di)methoxy-derivative(s)) of leoligin (e.g. 5-methoxy-leoligin) and/or leoligin. The fluid extract (i.e. the fluid phase of the obtained extract) may be concentrated taking advantage of routine techniques, some of which are exemplarily described herein below. Such concentration techniques include, but are not limited to fluidised-bed drying, concentration to a syrup or concentrated fluid extract, spray drying, freeze drying or the use of a vacuum dryer, a drying tunnel, vacuum band dryer or a drying hurdle. Often organic-hydrous fluid extracts (such as the fluid extract obtained herein using an organic solvent) are concentrated by nucleate boiling or surface evaporation.

Routine drying techniques employed in the pharmaceutical field comprise distillation and drying under normal conditions (i.e. room temperature) also methods which take advantage of variations in pressure and temperature in order to obtain the dried extracts. One well known method for preparing a dried extract is as follows: First, a fluid extract or tincture is prepared; after subsequent distillation of the solvent a viscous extract is obtained, to which often adjuvants and/or excipients (e.g. lactose, polyvinylpyrrolidone, sucrose, silicon dioxide and the like are added. This moist mass is then dried in suitable driers. Also employed in this context is the use of a vacuum band dryer (Mitchell Dryers Ltd), wherein a dried extract is obtained from the viscous extract after a pre-drying step using downdraft vaporizers.

Also envisaged herein is the use of commercially available extracts, in particular dried extracts, obtained from (a) plant(s) belonging to the genus Leontopodium.

After mechanical disruption of the cell(s), tissue(s) or whole plant(s) the plant material may be further macerated and/or dissolved/suspended in an organic solvent, such as hexane, petroleum benzene, chloroform, dichloromethane, acetone, ethyl acetate, diethyl ether, liquid carbon dioxide, ethanol and mixtures of water and alcohol with any of the solvents separately or subsequently with a second solvent or mixtures of different solvents. Preferably, dichloromethane and methanol are used as extraction solvents.

As shown in the appended examples, a hexane extract comprising 0.67% leoligin and 1.47% leoligin and its methoxy-derivative(s) can easily be prepared by routine techniques. However, it is preferred herein that the extract is enriched in the compounds described and provided herein, in particular compounds of formula (I), such as (di)methoxy-derivative(s)) of leoligin (e.g. 5-methoxy-leoligin) and/or leoligin. As also shown in the appended examples, higher yields (relative to the leoligin content [w/w %] in the extract) typically in a range between 0.7% to 1.5% can easily be obtained using standard extraction methods and solvents (such as dichloromethane). Using these standard extraction methods, yields of up to about 2.2% of leoligin and its 5-methoxy-derivative can be obtained. As described herein, the content of compounds of formula (I), such as (di)methoxy-derivative(s)) of leoligin (e.g. 5-methoxy-leoligin) and/or leoligin, can be further increased by multiple extraction rounds, e.g. a first extraction step using hexane followed by (a) subsequent extraction step(s) using e.g. dichloromethane, chloroform or ternary butyl methyl ether (=tBMe). A total lignan content (predominantly compounds of formula (I), such as (di)methoxy-derivative(s)) of leoligin (e.g. 5-methoxy-leoligin) and/or leoligin) of at least 2.4% can be achieved if subsequent extraction steps are applied The concentration of lignans (predominantly compounds of formula (I), such as (di)methoxy-derivative(s)) of leoligin (e.g. 5-methoxy-leoligin) and/or leoligin) can also be increased by the use of Sephadex-LH20-column chromatography (increase in the leoligin content from about 0.7% to about 2.2%).

Preferably, the extract is an enriched extract, i.e. contains compounds of formula (I), such as (di)methoxy-derivative(s)) of leoligin (e.g. 5-methoxy-leoligin) and/or leoligin in a high amount. Such an enriched extract can, for example, be obtained by taking advantage of silica gel chromatography as demonstrated in the appended examples. Silica gel column chromatography is well known in the art and described in detail in standard textbooks, such as “Preparative Chromatography Techniques” by Hostettmann, K. Marston, Andrew Hostettmann, Maryse, Springer-Verlag GmbH, 2007, 260 p. In the experimental section, it was shown that a pronounced increase in the leoligin content from 1.36% to 9.76% [w/w] can be achieved using silica gel chromatograph (mobile phase: petroleum ether-acetone).

Accordingly, it is preferred herein that the solid components of the extract (e.g. after evaporating the solvent by any of the drying methods described herein) comprise at least 0.05%, 0.1%, 0.5%, 0.7%, 1%, 1.5%, 2.0%, 2.5% or 3.0% of the compounds of formula (I), such as (di)methoxy-derivative(s)) of leoligin (e.g. 5-methoxy-leoligin) and/or leoligin, wherein an extract, the solid components of which comprise at least 0.7% of these compounds can be considered an “enriched” extract in context of the present invention. More preferably, the solid components of the extract comprise at least 5%, 6%, 7%, 8%, and most preferably at least 9% or 10% of the compounds of formula (I), such as (di)methoxy-derivative(s)) of leoligin (e.g. 5-methoxy-leoligin) and/or leoligin. An extract, the solid components of which comprise at least 9% of these compounds can be considered a “highly enriched” extract. An “enriched extract”, and, in particular a “highly enriched” extract as defined herein, represents therefore a preferred embodiment of the present invention. “Enriched” or “highly enriched” extracts are particularly useful in the herein disclosed medical context, in particular the stimulation of angiogenesis and/or treatment or prevention of hypovascularity, and/or further defined specific diseases and disorders. In accordance with the present invention, it is also preferred herein that the solid components of the (highly enriched) extract comprise at least 15%, 20%, 25%, 30%, 40%, 50%, 60%, 80% or 90% of the compounds (in particular compounds of formula (I), such as (di)methoxy-derivative(s)) of leoligin (e.g. 5-methoxy-leoligin) and/or leoligin. Based on the teaching provided herein a skilled person is readily in the position to determine whether an extract prepared in accordance with the present invention is enriched/highly enriched in compounds described in this invention (in particular compounds of formula (I), such as (di)methoxy-derivative(s)) of leoligin (e.g. 5-methoxy-leoligin) and/or leoligin. Most preferably, pure compounds of formula (I) are obtained, i.e. solid components of the extract comprise at least 95% of the compounds described and provided herein. In order to obtain a higher yield of the compounds of formula (I), the basic extracted material may be subjected to at least one further and up to eight further cycles of extraction. It is preferred that the (enriched/highly enriched) extract is obtained from (a) plant(s) belonging to the genus Leontopodium, in particular from the roots of such (a) plant(s). Exemplary species or cultivars of the above genus and to be used in accordance with the present invention are known in the art and also disclosed herein.

It is envisaged herein, that the “enriched/highly enriched” extract comprises predominantly (di)methoxy-derivative(s)) of leoligin (in particular 5-methoxy-leoligin) as active substance, in particular in combination with leoligin. Based on the teaching given herein, a skilled person is readily in the position to determine which amount of the (enriched/highly enriched) extract is to be employed in particular in the preparation of the pharmaceutical compositions comprising/consisting of the extract depending on the concentration/content of the herein disclosed active substance (preferably of compounds of formula (I), such as (di)methoxy-derivative(s)) of leoligin (e.g. 5-methoxy-leoligin) and/or leoligin and mixtures thereof). Preferably, the extract employed/contained in the pharmaceutical composition exerts substantially the same medical effect as a pharmaceutical composition comprising (a) (in particular compounds of formula (I), such as (di)methoxy-derivative(s)) of leoligin (e.g. 5-methoxy-leoligin) and/or leoligin. “Substantially the same effect” means in context of the present invention that the “effect” varies by less than 10%, preferably less than 5%, most preferably less than 1%. An exemplary “effect” to be measured is the stimulation of angiogenesis which may be reflected in capillary formation, capillary sprouting and/or increase in vascular density. In the context of wound healing, the effect may be migration of endothelial cells into the site of damage. The measurement of such effects as described herein above can easily be performed by a skilled person and is also demonstrated in the appended examples.

As mentioned above, the herein provided and disclosed extracts obtained from (a) plant(s) belonging to the genus Leontopodium can, in accordance with the present invention, be used in a medical context. It is preferred herein that the highly enriched extract predominantly comprises compounds of formula (I), such as (di)methoxy-derivative(s)) of leoligin (e.g. 5-methoxy-leoligin) and/or leoligin. Formulas of leoligin and preferred (di)methoxy-derivatives thereof are also provided herein. The herein disclosed pharmaceutical composition comprising a (root) extract obtained from a plant belonging to the genus Leontopodium, wherein the extract is preferably enriched (most preferably highly enriched) in the compounds of formula (I), such as (di)methoxy-derivative(s)) of leoligin (e.g. 5-methoxy-leoligin) and/or leoligin, is used in the treatment or prevention of hypovascularity and/or in stimulating angiogenesis. The term “root extract” used herein means an extract obtained from roots, i.e. plant material from the lower parts of the plants are used, preferably only roots are used as raw plant material in the preparation of the extract. It is preferred in this context that the pharmaceutical composition consists of the (preferably enriched, more preferably highly enriched) extract. However, further excipients/adjuvants/carriers and the like as described herein and known in the art may be contained in the pharmaceutical composition in addition to the extract. In accordance with the above, a composition comprising (consisting of) a(n) (root) extract obtained from a plant belonging to the genus Leontopodium, whereby the extract is (highly) enriched in compounds of formula (I), such as (di)methoxy-derivative(s)) of leoligin (e.g. 5-methoxy-leoligin) and/or leoligin (or mixtures thereof), is provided herein for use in medicine or for use as a medicament. Also a (root) extract obtained from a plant belonging to the genus Leontopodium, whereby the extract is (highly) enriched in compounds of formula (I), such as (di)methoxy-derivative(s)) of leoligin (e.g. 5-methoxy-leoligin) and/or leoligin for use in medicine or as a medicament is provided. It is envisaged that the above (pharmaceutical) compositions/extracts are to be used in accordance with the present invention in the stimulation of angiogenesis and/or the treatment or prevention of hypovascularity and/or the treatment or prevention of further herein defined specific diseases/disorders.

In order to obtain the single compounds, the extracts may be prepared and evaporated as described above and, submitted to further purification by column chromatography using silica gel, silica gel modified by means of AgNO₃, reversed phase material (RP18) or Sephadex LH 20® as stationary phases. Additionally, other separation techniques e.g. high speed counter current chromatography or (semi)-preparative HPLC might be used as well. Fractions obtained by the above mentioned chromatographic techniques may be further purified, e.g. by another cycle of chromatographic purification. For example, a cross-linked dextran gel may be used for such further purification, like e.g. Sephadex LH-20®. This kind of chromatography is usually performed in the presence of an organic solvent such as methanol, acetone dichloromethane and the like. It is envisaged herein that the herein described pharmaceutical compositions comprising the extract disclosed herein may also (in addition) comprise the pure (and/or (substantially) purified, e.g. purified from the extract) active substances (i.e. compounds of formula (I), such as (di)methoxy-derivative(s)) of leoligin (e.g. 5-methoxy-leoligin) and/or leoligin. In accordance with the above, it is preferred herein that the pharmaceutical composition comprises essentially the plant extracts disclosed herein and obtained by the herein described methods. Also envisaged herein is a pharmaceutical composition, which does not comprise the herein described extract, but comprises the pure (and/or (substantially) purified, e.g. purified from the extract) active substances (i.e. compounds of formula (I), such as (di)methoxy-derivative(s)) of leoligin (e.g. 5-methoxy-leoligin) and/or leoligin). The extract can also be obtained by alternative extraction methods known in the art e.g. supercritical carbon dioxide extraction, percolation or Soxhlet-extraction and adaptable for the means and methods of the present invention by one skilled in the art.

Also envisaged herein, though less preferred, the compounds may also be obtained from upper parts of the plants, e.g. flowers, stems, leaves, seeds and the like. Leontopodium alpinum (Edelweiss) plants to be extracted are easily available e.g. from the Station féderale de recherches en production vegetale de Changins (see also http://www.admin.ch/sar/rac; Revue Suisse Vitic. Arboric. Hortic. 31(2), 889-96 (1999)).

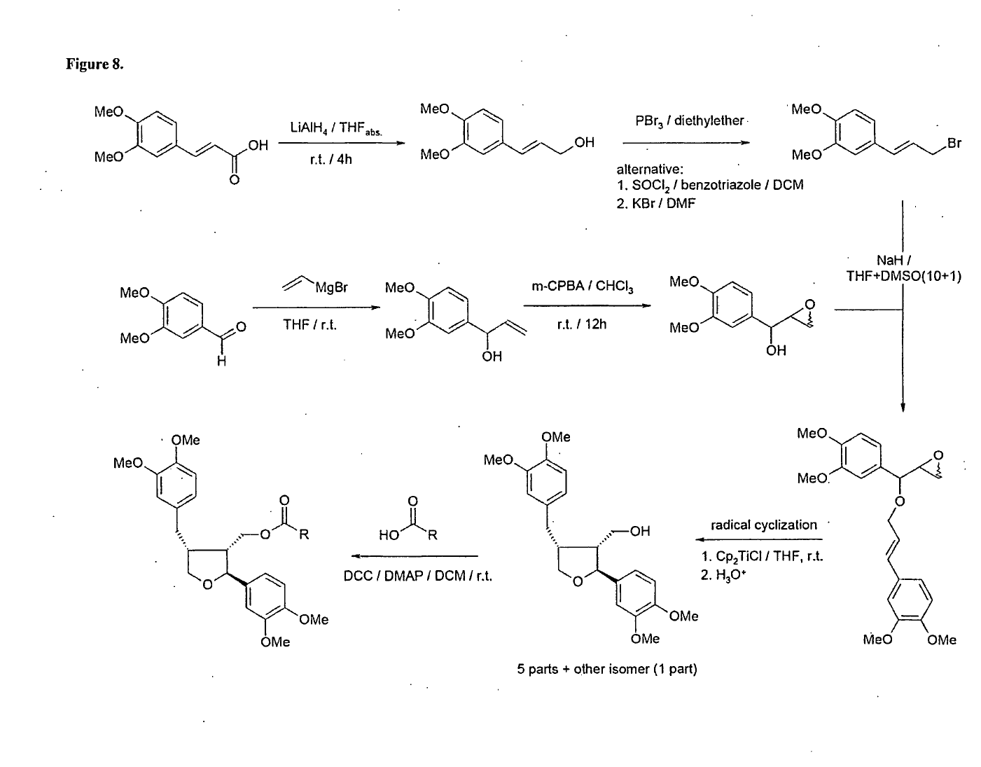

In an alternative embodiment, the compounds to be used herein may also be synthesized. An exemplary synthetic pathway of leoligin is shown in FIG. 8. The shown synthetic pathway might be adapted by a change of the corresponding educts to obtain other compounds of the present invention, in particular compounds of formula (I), such as (di)methoxy-derivative(s)) of leoligin (e.g. 5-methoxy-leoligin). A skilled person will be aware of methods of synthesizing the compounds of the present invention, such as (di)methoxy-derivative(s)) of leoligin (e.g. 5-methoxy-leoligin) or leoligin, or may deduce corresponding methods e.g. from Li Hong Hu, J. Nat. Prod. 68, 342-8. (2005); Babasaheb P. Bandgar, Monatshefle für Chemie 135, 1251-5 (2004); J Pijus Kumar Mandal, Org. Chem. 63, 2829-34 (1998); Subhas Chandra Roy, J. Org. Chem. 67, 3242-8 (2002).

As mentioned above, the active compounds referred to herein may also be provided via semi-synthetic methods, e.g. by derivatizing a natural product such as leoligin or its methoxy-derivatives (e.g. 5-methoxy-leoligin). Suitable derivatization reactions known in the art comprise methods wherein the ester bond present in leoligin is saponified to produce an alcohol. The alcohol may be oxidized to provide a carbonyl]carboxylic acid functionality to be reacted with an alcohol, thiol or amine, or it may be esterified with a different organic acid, it may be converted into an amine etc.

The pharmaceutical composition may comprise the compounds provided in the present invention. The compounds to be used in accordance with the present invention may be obtained from Leontopodium plants as described herein above and/or chemically synthesized.

The pharmaceutical composition of the present invention comprising compounds of formula (I) and, in particular, (di)methoxy-derivative(s)) of leoligin (e.g. 5-methoxy-leoligin) and/or leoligin, will be formulated and dosed in a fashion consistent with good medical practice, taking into account the clinical condition of the individual patient, the site of delivery of the pharmaceutical composition, the method of administration, the scheduling of administration, and other factors known to practitioners. The “effective amount” of the pharmaceutical composition for purposes herein is thus determined by such considerations.

The skilled person knows that the effective amount of pharmaceutical composition administered to an individual will, inter alia, depend on the nature of the compound. For example, if said compound is a lignan the total pharmaceutically effective amount of pharmaceutical composition administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day. If given continuously, the pharmaceutical composition is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect. The particular amounts may be determined by conventional tests which are well known to the person skilled in the art.

As pointed out above, pharmaceutical compositions comprising compounds of formula (I) (and derivatives thereof) should be employed in non-invasive conservative therapy, i.e. they may be administered parenterally, orally, rectally, intracisternally, intravaginally, intraperitoneally, topically (as by powders, ointments, drops or transdermal patch), bucally, or as an oral or nasal spray. Accordingly, also the compound provided herein may be administered by any one of a parenteral route, oral route, intravenous route, intraarterial route, intramuscular route, intracardial route, intrapulmonal route, intravesical route, intravitreal route, (sub-)cutaneous route, intranasal route or transdermal route. It is preferred herein that the herein described compounds of formula (I) are applied locally, e.g. via injection (inter alia via a syringe) into (an) affected tissue(s)/organ(s). Such a local application/injection may be performed via a catheter-based application. Under certain circumstances the application of depot injectables may be indicated, optionally also (in addition) systemic administration. For local application, the herein described compounds may be applied via (wound) dressings, plasters, etc. The compounds may be also be contained in such dressings, plasters or applied to these dressings/plasters for example as ointment or liquid or any other appropriate form. Local administration may also be performed when the pharmaceutical composition described herein is in liquid form (e.g. eye drops and the like). Preferably, conservative application/therapy is indicated in the administration of the herein described compounds of formula (I) and its derivatives, like leoligin and 5-methyl-leoligin.

Pharmaceutical compositions to be used in accordance with this invention preferably comprise a pharmaceutically acceptable carrier. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The pharmaceutical composition is also suitably administered by sustained release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained release pharmaceutical compositions also include liposomally entrapped compounds. Liposomes containing the pharmaceutical composition are prepared by methods known per se: DE 3,218,121; Epstein et al., Proc. Natl. Acad. Sci. (USA) 82:3688-3692 (1985); Hwang et. al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, the pharmaceutical composition is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation.

Generally, the formulations are prepared by contacting the components of the pharmaceutical composition uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non aqueous vehicles such as fixed oils and ethyl oleate are also useful herein, as well as liposomes. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The components of the pharmaceutical composition to be used for therapeutic administration must be sterile. Sterility is readily accomplished by filtration through sterile filtration membranes (e.g., 0.2 micron membranes). Therapeutic components of the pharmaceutical composition generally are placed into a container having a sterile access port, for example, an intravenous solution bag or vial having a stopper pierceable by a hypodermic injection needle.

The components of the pharmaceutical composition ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized compound(s) using bacteriostatic Water-for-Injection.

The present invention is further described by reference to the following non-limiting figures and examples.

The Figures show:

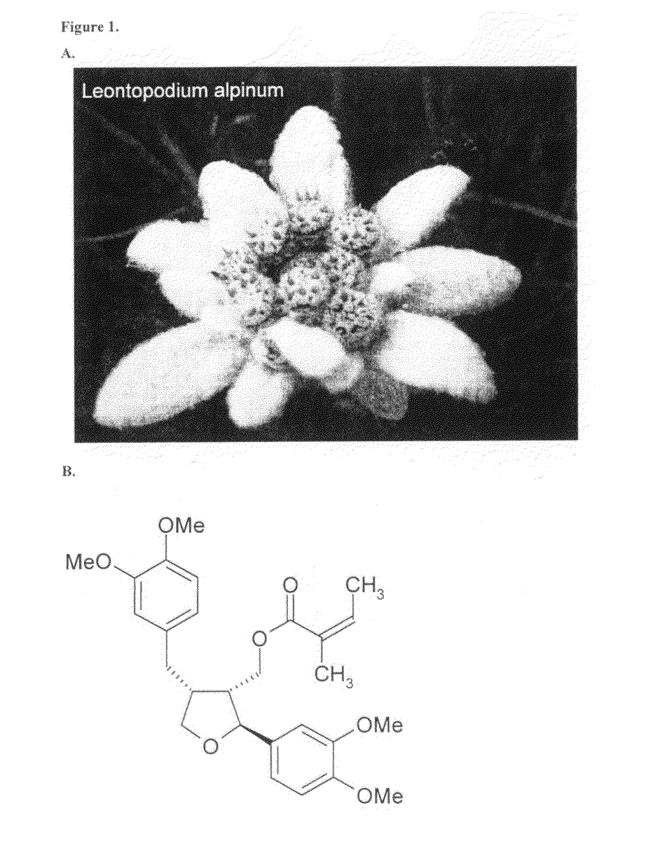

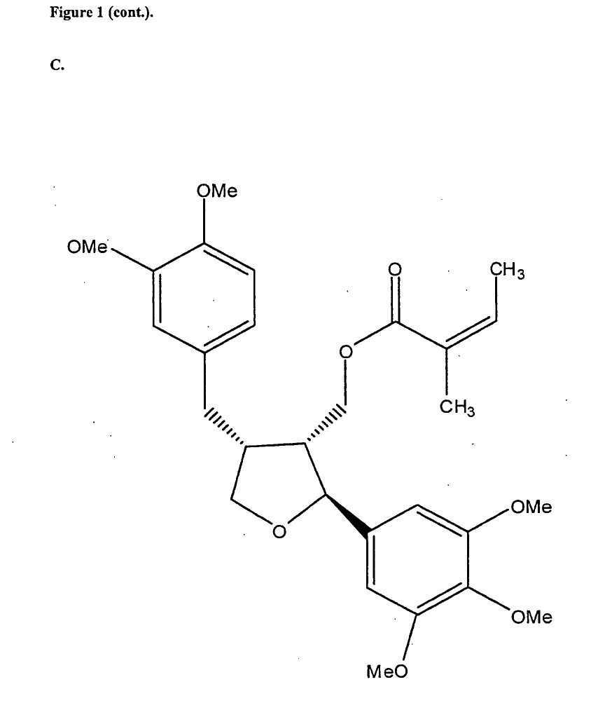

FIG. 1. Leoligin and 5′-methoxy-leoligin are constituents of Edelweiss (Leontopodium alpinum Cass.) roots

Edelweiss is one of the most popular alpine plants and is also used in folk medicine for the treatment of indigestion, fever, and “abdominal aches”. FIG. 1A shows the flower of Edelweiss (Leontopodium alpinum Cass.). FIG. 1B shows the chemical structure of leoligin—IUPAC name: [(2S,3R,4R)-4-(3,4-dimethoxybenzyl)-2-(3,4-dimethoxyphenyl)tetrahydrofuran-3-yl]methyl (2Z)-2-methylbut-2-enoat. FIG. 1C shows the chemical structure of 5′-methoxy-leoligin (LaG2)—IUPAC name: [(2S,3R,4R)-4-(3,4-dimethoxybenzyl)-2-(3,4,5-trimethoxyphenyl)tetrahydrofuran-3-yl]methyl-(2Z)-2-methylbut-2-enoat. 5′-methoxy-leoligin (LaG2) has a molecular weight of 500.59, an exact mass of 500. The molecular formula is C₂₈H₃₆O₈ and the molecular composition is C 67.18%, H 7.25% and O 25.57%.

Leoligin and 5′-methoxy-leoligin (LaG2) are lignans, which were isolated from the roots of Edelweiss (Leontopodium alpinum Cass.).

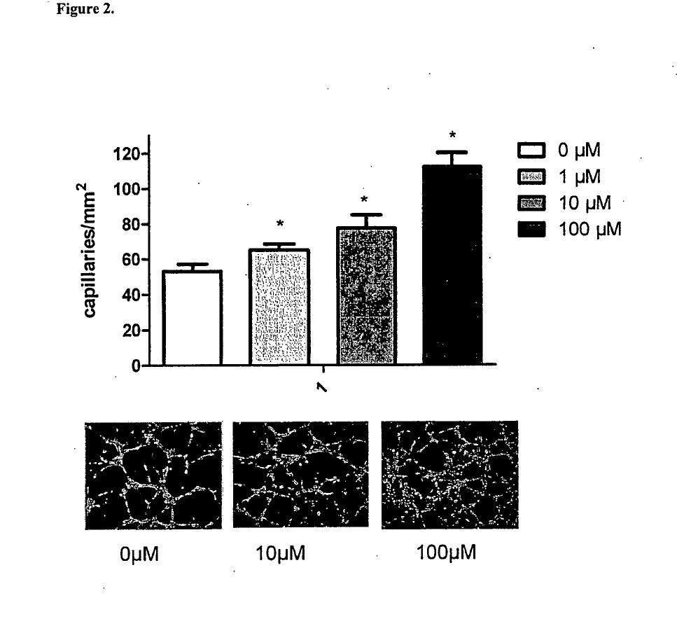

FIG. 2. 5-methoxy-leoligin (Lag2) promotes endothelial tube formation, a process that induces angiogenesis

FIG. 2 shows the induction of capillary formation of human microvascular endothelial cells on matrigel substrate. Cells form tube-like structures between each other within 6 hrs of incubation. Those were quantified by an imaging software. LaG2 dose-depentently supported capillary formation.

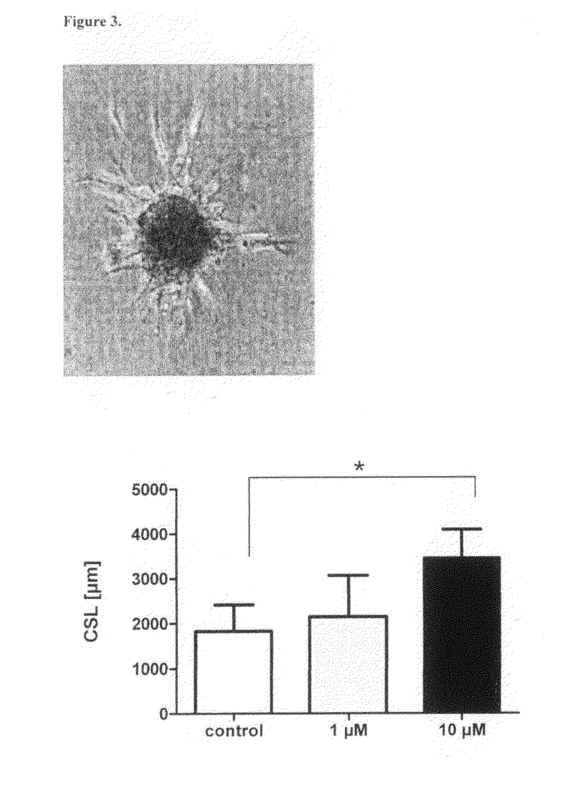

FIG. 3. Effects of 5-methoxy-leoligin (Lag2) stimulates angiogenic sprouting

FIG. 3 shows angiogenic sprouting in a 3-D Collagen matrix. The left image shows a cell spheroid of HUVECs with characteristic angiogenic sprouts. These sprouts were analyzed after 24 h of stimulation and “cumulative sprout length” (CSL) of each sprout was quantified in μm. The application of LaG2 led to a significant increase of sprout number and lengths.

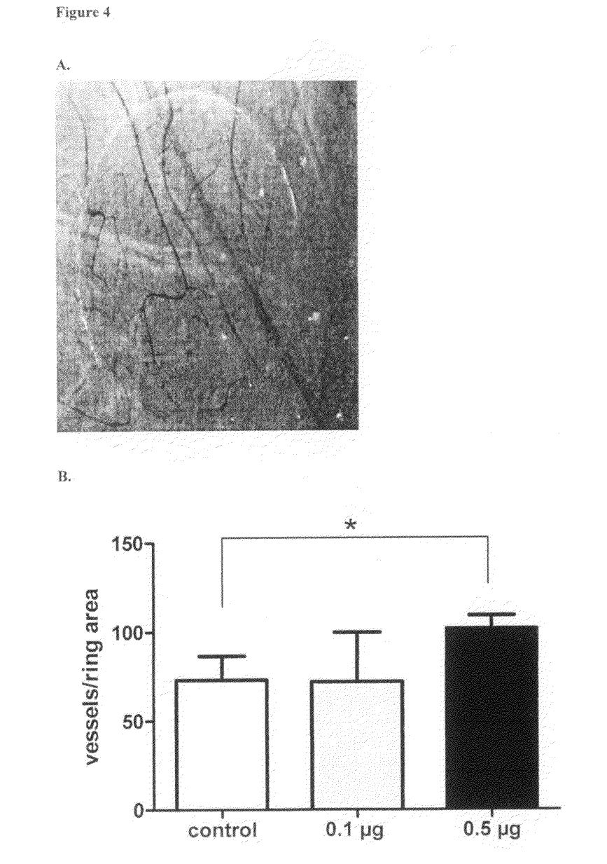

FIG. 4. 5-methoxy-leoligin (Lag2) increases blood vessel density in the chicken chorioallantoic membrane assay

FIG. 4 shows the analysis of blood vessel formation in the chicken chorioallantoic membrane assay. The image on the left shows a typical chicken embryo with blood vessels and a Permanox® ring for application of drugs. LaG2 dose-dependently supported embryonic blood vessel formation. stars indicate p<0.05. Each group consists of 5 eggs.

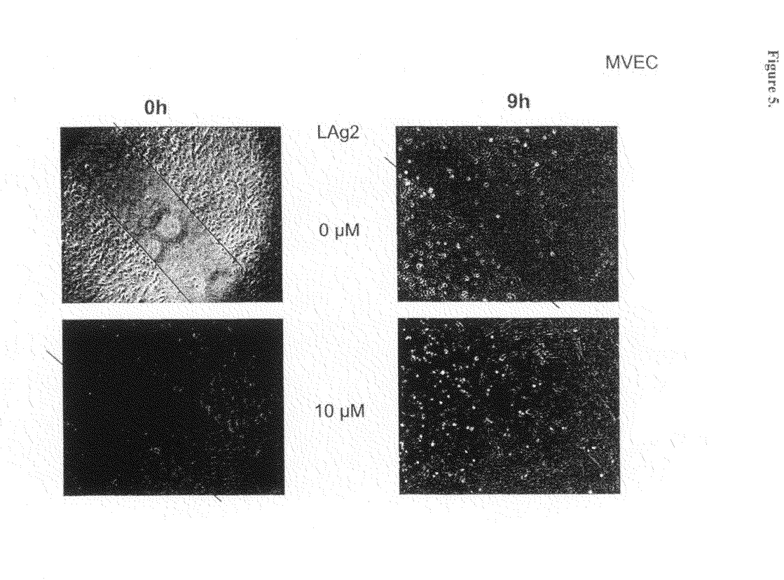

FIG. 5. 5-methoxy-leoligin promotes the migration of endothelial cells into damaged tissue and stimulates wound healing.

FIG. 5 shows a wound scratch healing assay. The upper two images show the migration of endothelial cells into scratch area without treatment (left side) an in the presence of Lag2 (right side). The two images at the bottom are the control images of the scratch size at 0 hours.

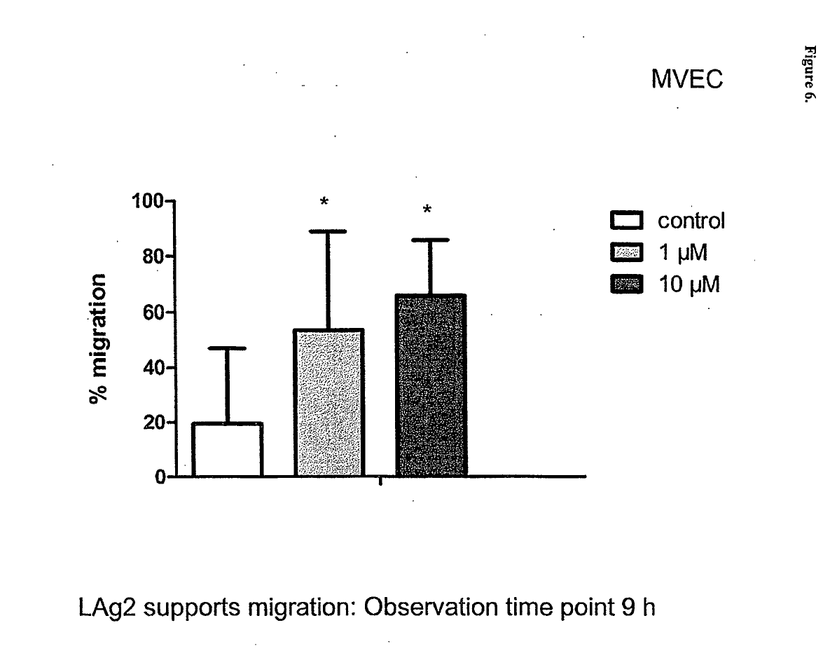

FIG. 6. Quantification of wound healing induced by 5-methoxy-leoligin

FIG. 6 displays quantitative data of a wound scratch healing assay of untreated cells and cells treated with 1 and 10 μM of Lag2 after 9 hours of incubation

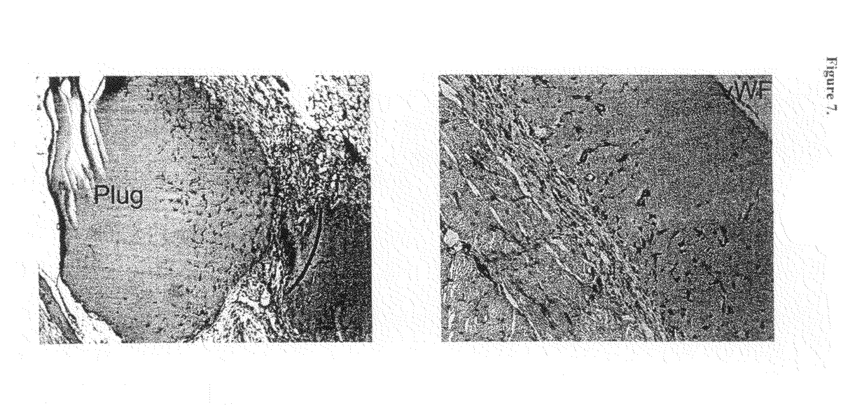

FIG. 7. Matrigel Plugs in C57BL/6 mice. Lag2 was injected into matrigel plugs for 10 days of in vivor growth (15 ng/ml bFGF). Blood vessels and lymphatics will be stained with LYVE-1 and vWF. Assay has been established for bFGF induced revascularization.

FIG. 8. Pathway

FIG. 8 shows a possible synthetic pathway of leoligin.

FIG. 9. Wound healing induced by topical administration of leoligin FIG. 9 shows time to wound closure in a topical application porcine model of wound closure. N for control and Leoligin treated wounds is 5. Data show mean values +/−SEM. Data assessment was conducted in a blinded manner. Data indicate time (in days) until wound closure after surgery.

The following Examples illustrate the invention.

### EXAMPLE 1

### Material and Methods

### General

All reagents used were of purissimum or analytical grade quality and were purchased from Sigma Aldrich (Sigma-Aldrich, Vienna, Austria) if not specified otherwise. Water was produced by reverse osmosis followed by distillation.

### Plant Material, Isolation, and Purification of Leoligin

Ground roots (1907.84 g) from L. alpinum Cass. were exhaustively macerated with dichloromethane (12.5 l DCM, at RT, eight times). Voucher specimens are deposited at the herbarium of the Institut für Pharmazie/Pharmakognosie, Leopold-Franzens-Universität Innsbruck. Extracts were evaporated to dryness yielding 43.0 g crude dichloromethane extract. 40.0 g of the obtained crude extract were redissolved in 100 ml MeOH and separated in a MeOH soluble and insoluble part. The soluble part was separated by Sephadex LH 20 (Pharmacia Biotech, Sweden) CC (90×3.5 cm) with MeOH as mobile phase yielding 8 fractions. Fraction 5 (15.13 g; 320-410 ml elution volume) was rechromatographed by silica CC (180 g, 41×3.5 cm) using a PE-acetone gradient with an increasing amount of acetone yielding 40 fractions (A-1 to A-40). A small amount (28.2 mg) of Fraction A-21 (PE/acetone, 85:15; 441.2 mg) was separated by semi preparative HPLC (Phenomenex Synergy Max-RP column (10 μm, 10×250 mm); 55% acetonitrile/45% water, isocratic; flow: 3.50 ml/min; 25° C.) yielding 16.0 mg pure leoligin and i4.0 mg of its 5-methoxy derivative. A small amount (26.5 mg) of Fraction A-22 (PE/acetone, 85:15; 77.8 mg) was separated by semi preparative HPLC (Waters X-Terra Prep MS C18, 5 μm, 7.8×100 mm column; 70% MeOH/30% water, isocratic; flow: 1.50 ml/min; 25° C.) yielding 6.3 mg of the 5,5′-dimethoxyderivative of leoligin.

Alternatively, leoligin and its derivates (for example 5-methoxy-leoligin were isolated and purified as follows:

Ground roots (5.30 kg) from L. alpinum Cass. were exhaustively macerated with dichloromethane (15.0 L DCM, at RT, nine times). Voucher specimens are deposited at the herbarium of the Institut für Pharmazie/Pharmakognosie, Leopold-Franzens-Universität Innsbruck. Extracts were evaporated to dryness yielding 84.23 g crude dichloromethane extract. 60.0 g of the obtained extract were mixed with the same amount of silica gel and applied on the top of a wet silica gel column (prepared in pure petrol ether; ø=10 cm, high=40 cm; 600 g silica gel). The column was eluted with mixtures of petrol ether with increasing amounts of acetone. The eluat was collected in portions of 20 mL. Fractions 308 to 376 (eluted with petrol ether/acetone 8+2 (v/v) and 7+3 (v/v)) contained leoligin and its derivatives. The obtained fractions were combined and evaporated to dryness yielding 7.00 g of an enriched fraction. This fraction was further purified by means of Sephadex LH 20 column chromatography with methanol as mobile phase followed by Sephadex LH 20 CC using dichloromethane/acetone (85+15, v/v) as mobile phase yielding a highly enriched fraction of 545.57 mg. The further separation was performed with 282 mg of the enriched fraction by repeated high speed counter current chromatography (HSCCC) using a mixture of water, ethyl acetate, n-hexane and methanol (1+1+1+1.5, v/v/v/v) using the lower phase as mobile phase and the upper layer as stationary phase (tail to head modus). The used coil volume was 230 mL with 800 rpm and a flow rate of 1.0 mL/min. The sample was dissolved in 0.5 mL upper and 0.5 mL lower phase and injected in the equilibrated system. The eluate was collected in fractions of 5 mL. The separation afforded 12.2 mg 5,5′-dimethoyxleoligin (fraction 11 and 12); 45.6 mg pure 5-methoyxleoligin (fraction 50-55); 109.0 mg of a mixture of leoligin and 5-methoyxleoligin (fraction 56-60) as well as 50.1 mg pure leoligin (fraction 61-65). The mixture of leoligin and 5-methoyxleoligin (fraction 56-60) was used for a further separation using identical HSCCC parameter to maximize the yield of the lignan derivatives resulting in 27.2 mg 5-methoyxleoligin and 81.7 mg leoligin.

### Preparation of Extracts Enriched in Leoligin and its Methoxy-Derivative

In order to quantify the content of leoligin and its methoxy-derivative in different extract preparations several extraction procedures were used. Therefore ground roots (20.00 g) from L. alpinum Cass. were exhaustively macerated with dichloromethane (100 ml DCM, at RT, eight times). After filtration the obtained extracts were combined, and evaporated to dryness to yield a semi solid DCM-extract. Other extracts were prepared by ultrasonic extraction using 20.00 g ground roots which were sonicated for 15 min using 1×200 ml and 1×100 ml of solvent or a second ultrasonic extraction cycle after air drying of the plant material (5.00 g; 2×15 min; 2×100 ml solvent). The leoligin content was determined by means of HPLC-quantification using the method of external standard. Each extract was prepared in duplicate and quantified in triplicate. The content of 5-methoxy-leoligin was calculated using the calibration curve of leoligin.

### Results

### Preparation of Extracts Enriched in Leoligin and its Methoxy-Derivates

Root extracts comprising leoligin and its derivates 5-methoxy-leoligin and 5,5′-dimethoxy-leoligin have been prepared as described herein above (i.e. roots macerated at room temperature and extracted using dichlormethane), whereby the yield of the extract lies typically in the range of between 1.03 bis 2.26% and whereby the maximum level of the leoligin and methoxy-leoligin content (quantified as a mixture thereof) is 2.14%.

In order to obtain extracts enriched in leoligin and its derivates, the plant material is in a first step extracted with hexane or heptane followed by a subsequent extraction with organic solvents dicholoromethane, chloroform or ternary butyl methyl ether. The results are summarized in the table below.

| | | | | Total lignan |
|---|---|---|---|---|
| Pretreatment | | Yield of | Leoligin | content in the |
| solvent | Extraction | extract | content (w %) | extract (calculated |
| (defatting) | solvent | (w %) | in the extract | as leoligin; w %) |
| — | dichloromethane* | 1.03-2.26% | 0.77-1.36% | 1.69-2.14% |
| — | n-hexane** | 0.07-0.15% | 0.67% | 1.47% |
| — | n-heptane** | 0.12-0.27% | 0.74% | 1.55% |
| n-hexane | dichloromethane*** | 0.50% | 1.32% | 2.67% |
| n-hexane | chloroform*** | 0.65% | 1.31% | 2.71% |
| n-hexane | tBMe*** | 0.39% | 1.19% | 2.43% |

*exhaustive mazeration (20.00 g; 8x100 ml);
**ultrasonic extraction (20.00 g; 15 min; 1x200 ml; 1x100 ml);
***ultrasonic extraction (5.00 g; 2x15 min; 2x100 ml).

“Yield of extract” refers to the weight of the extract vs. the basic material used, whereas leoligin content refers to the percentage by weight of Leoligin and lignan, respectively, in the extract.

The concentration of lignans (and derivatives) was also increased by the use of Sephadex-LH20-column chromatography (increase in the leoligin content from 0.77% to 2.21%). The most pronounced increase (increase in the leoligin content from 1.36% auf 9.76%) was achieved using silica gel column chromatograph (mobile phase: Petroleum ether-aceton).

### EXAMPLE 2

### Hind Limb Ischemia Model in the Mouse

Male C57BL/6 wild-type mice at the age of 12-18 months are subjected to unilateral hind-limb surgery under anesthesia with intra-peritoneal administration of ketamine (90 mg/kg) and xylazine (12 mg/kg). Briefly, the left femoral artery was exposed, ligated with 5-0 silk ligatures, and excised. For therapy mice are injected with the compound (methoxy-leoligin (100 μl of a 100 μM, 10 μM and 1 μM of LAg2 or control) into thigh and calf muscles immediately after surgery. Lag2 is dissolved in a total volume of 300 μl saline and injections of 100 μl are to be performed at 2 sites of the thigh and at 1 site of the calf.

Blood flow measurement, in vivo assessment of limb function, and ischemic damage blood flow measurements are performed using a laser Doppler perfusion image (LDPI) analyzer (Moor Instruments, USA). To minimize data variables attributable to ambient light and temperature, animals are kept on a heating plate at 37° C. before measurement, and blood perfusion is expressed as the LDPI index representing the ratio of left (═Operated, ischemic leg) versus right (=not-operated, not-ischemic leg) limb blood flow. A ratio of 1 before operation indicates equal blood perfusion of both legs, whereas after femoral artery excision this ratio usually drops to 0.27, indicating severe attenuation of leg blood supply in the operated leg. Blood flow is displayed as changes in the laser frequency, represented by different colour pixels. Assessment of impaired use of the ischemic limb is performed on days 7, 14, 21 and 28 according to a movement score described in the literature (f=use of the leg, 2=active foot use, 3=fu11 use of foot without spreading of toes, 4=unrestricted movement). Determination of ischemic tissue damage and loss is performed on days 7, 14, 21 and 28 after induction of hind-limb ischemia. Mice are considered as positive for having necrosis when at least one toe is necrotic.

### Immunofluorescence

For tissue staining, mice are sacrificed after 4 weeks and ischemic limb tissues are retrieved. Specimens are fixed in 10% (v/v) buffered formaldehyde, dehydrated with graded ethanol series, and embedded in paraffin. Alternatively, fresh tissue is embedded in OCT compound (TISSUE-TEK®, Sakura Finetek) and snap-frozen in liquid nitrogen. Tissues are sliced into 5-μm sections. For determination of capillary density, vascular endothelial cells are identified by immunohistochemical staining for CD31 (Pharmingen), and for assessment of artery/arteriole density sections are stained with a mouse monoclonal alpha-smooth muscle actin antibody (Pharmingen). For fluorescent microscopy, appropriate secondary antibodies (Alexa 488 for SMA and Alexa 594 for CD31; both from Invitrogen 1:200) are used. Adductor muscle samples of each leg are divided into 2 parts and capillaries (CD-31 positive) and arterioles (alpha-smooth muscle actin positive cells surrounding the whole circumference of the vessel) are counted in five sections of each part and are expressed as capillary and arteriole density per mm2.

For cell proliferation and apoptosis assays, after 7 days sections of muscles subjected to hind-limb ischemia treated by Lag2 therapy or saline are stained by Ki67 (polyclonal Rabbit Abeam antibody 15580) and subjected to the TUNEL assay from Roche (In Situ Cell Death Detection Kit, Fluorescein) as described by the manufacturer.

### EXAMPLE 3

### Myocardial Infarction Model in the Rat

MI is induced in 8- to 10-week old male rnu/rnu rats (Harlan Winkelmann, Borchen, Germany) through left anterior descending coronary artery (LAD) ligation. Rats included in the study weight 200-300 g. All animals receive humane care in compliance with the “Principles of Laboratory Animal Care”, formulated by the National Society for Medical Research, and the “Guide for the Care and Use of Laboratory Animals”, prepared by the Institute of Laboratory Animal Resource and published by the National Institutes of Health. Two weeks after ligation of the LAD, animals are re-operated to inject LAg2 into the myocardium. Several injections (˜20 μl each) are placed into and around the ischemic scar (groups are 100 μM, 10 μM and 1 μM of LAg2 or control (each n=20). Functional Assessment by Transthoracic Echocardiography Transthoracic echocardiography is performed before and 2 days after MI, as well as before re-surgery, and 2 weeks and 4 weeks after LAG2 injection with a Hewlett Packard SONOS 5500 Echocardiography system (Hewlett Packard, Andover, Mass., USA), using a commercially available high-frequency linear-array transducer system.

### Tissue Preparation for Morphologic Studies

Rats are killed humanely, hearts are harvested, and fibrous tissues removed. After intracardiac blood is rinsed away, the hearts are divided into 3 parts of equal thickness representing the base, middle, and apex of the heart. Each is snap-frozen in liquid nitrogen after being embedded in optimal cutting temperature compound (Tissue-Tec OCT Compound, Miles Inc, Elkhart, 1N, USA). From each part, 5 μm slides were prepared using a cryostat. Standard hematoxylin-eosin staining is performed to permit morphologic assessment.

### Statistical Analysis

Data are analyzed retrospectively using SPSS 15.0 for windows (Chicago, Ill., USA). Continuous variables are shown as mean with standard deviation. For comparison of control animals and animals treated with expanded or non-expanded UCB-HPCs we use the Mann-Whitney U-test. P values <0.05 were regarded as statistically significant.

### EXAMPLE 4

### Wound Healing Induced by Leoligin

### Porcine Model for Time of Wound Closure Analysis

For the large animal model 30-35 kg pigs (Deutsches Landschwein) was used. Animals were supplied by, kept, and experiments were performed at the Central Animal Testing Facility of the University of Innsbruck and the “Besondere Einrichtung für Biomedizinische Forschung” of the Medical University of Vienna. Pigs received a standard growth diet.

### Anaesthesia

One hour prior to anaesthesia pigs received an intramuscular injection of 4 mg/kg Azaperon and 0.1 mg/kg Atropin. Full anaesthesia was achieved by intravenously applied 2-3 mg/kg Propofol and 15 mg Piritramid, followed by intubation of animals and ventilation with 30% O₂. Muscle relaxation was achieved by an initial does of 8 mg Pancuronium and repetition with 0.2 mg/kg/h Pancuronium. Maintenance of anaesthesia was achieved by continuous infusion with 8-12 mg/kg/h Propofol and 15 mg Piritramid.

### Surgical Procedure

In the course of an experimental bypass surgery in the pig, after re-implantation of the graft (into the carotis artery) Leoligin (“Leo”) or NaCl control solution was added into the wound topically (the situs was filled with control solution (0.9% NaCl) or a solution containing leoligin at 100 μM. By optical inspection every 24 hours of blinded researchers the time to wound closure/healing was documented. Animals with infected wounds were excluded from the analysis.

The results as shown in the following table and in FIG. 9 demonstrate that compounds of formula (I) as described herein, such as the exemplary compound leoligin, can successfully be used to promote wound healing.

| | Control | Leo |
|---|---|---|
| | 19 | 19 |
| | 22 | 20 |
| | 24 | 16 |
| | 30 | 21 |
| | 26 | 21 |
| mean value | 24.2 | 19.4 |
| standard deviation | 4.14728827 | 2.07364414 |
| SEM | 1.88513103 | 0.94256552 |

1. A pharmaceutical composition comprising a compound of formula (I) 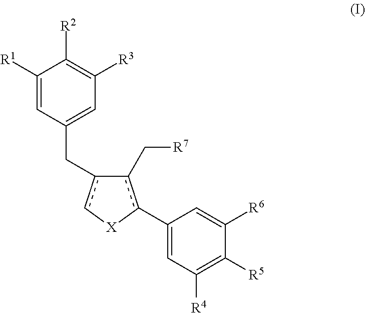
for stimulating angiogenesis and/or the prevention and/or treatment of an angiogenic disorder,
wherein
R¹, R² and R³ are independently selected from H, OH, halogen, alkyl, or alkoxy; and
R⁴, R⁵ and R⁶ are independently selected from H, OH, halogen, alkyl, or alkoxy;
R⁷ is selected from —OR⁸, —N(R^{8′})R⁸, —SR⁸, —C(O)R⁸, —OC(O)R⁹, —C(O)OR⁹, —N(R^{9′})C(O)R⁹, —C(O)N(R^{9′})R⁹ or —S(O)R⁹; wherein R⁸ and R⁹ are independently selected from alkyl or alkenyl and R^{8′} and R^{9′} are independently selected from H, alkyl or alkenyl; and wherein any alkyl or alkenyl group comprised in R⁷ may be unsubstituted or substituted by one or more substituents, selected from OH, halogen or alkoxy; and
X is selected from O, S, C(R¹⁰)R¹⁰ and NR¹⁰, wherein R¹⁰, independently for each occurrence, is H, alkyl or alkenyl;
and the dashed lines in the ring structure containing the group X indicate that the respective bond may be a single or a double bond;
or any pharmaceutically acceptable salt or solvate thereof.

2. A method for stimulating angiogenesis and/or the prevention and/or treatment of an angiogenic disorder, comprising the administration of a compound as defined in claim 1 to a subject in need thereof.

3. The pharmaceutical composition of claim 1 or the method of claim 2, wherein the compound of formula (I) has the stereochemistry indicated in formula (Ia): 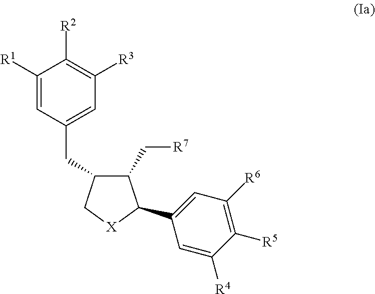
wherein R¹ to R⁷ and X are defined as in claim 1.

4. The pharmaceutical composition of claim 1 or 3 or the method of 2 or 3, wherein R⁷ is —OC(O)R⁹ or —C(O)OR⁹.

5. The pharmaceutical composition of any of claims 1, 3 and 4 or the method of any of claims 2 to 4, wherein at least one of R¹, R² and R³ and at least one of R⁴, R⁵ and R⁶ is an alkoxy group.

6. The pharmaceutical composition of any of claims 1, 3 and 4 or the method of any of claims 2 to 4, wherein at least two of R¹, R² and R³ and at least two of R⁴, R⁵ and R⁶ are alkoxy groups.

7. The pharmaceutical composition of claim 1 or the method of claim 2, wherein the compound of formula (I) has the following structure:

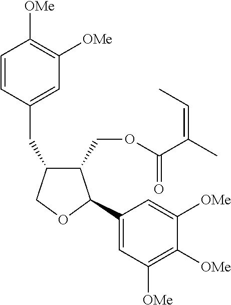 [SMILES: C/C=C(/C)C(=O)OC[C@H]1[C@@H](Cc2ccc(OC)c(OC)c2)CO[C@@H]1c1cc(OC)c(OC)c(OC)c1]

8. A pharmaceutical composition comprising a compound as defined in any of claims 1 to 7 for use in treating or preventing hypovascularity and/or the prevention and/or treatment of an angiogenic disorder.

9. A method for treating or preventing hypovascularity and/or the prevention and/or treatment of an angiogenic disorder in a subject suffering from or being prone to suffering therefrom, comprising the administration of a compound as defined in any one of claims 1 to 7 to said subject.

10. The method of any of claims 2 to 7 and 9, wherein said subject is a human.

11. The pharmaceutical composition of any of claims 1 and 3 to 8 or the method of any of claims 2 to 7, 9 and 10, wherein the stimulation of angiogenesis and/or the prevention and/or treatment of an angiogenic disorder comprises revascularisation during or after wound healing and/or during scar tissue formation of skin or inner organs, revascularisation after or during amputation, trauma, surgery and/or transplantation of tissues or organs, (re-)activation of vessel growth after or during necrosis, and/or wherein the prevention or treatment of hypovascularity and/or the prevention and/or treatment of an angiogenic disorder comprises the prevention or treatment of a disease/disorder selected from the group consisting of non-diabetic retinopathy, vascular obliteration, arterio- and veno-microvasculopathy, arterio- and veno-microangiopathy, ischemic disorders, non-cardial ischemic diseases or disorders, disorders relating to hibernating tissue, stable angina abdominalis, vascular dementia and penile dysfunction.

12. The pharmaceutical composition of claim 11 or the method of claim 11, wherein said ischemic disorder is selected from the group consisting of myocardial infarction and stroke.

13. The pharmaceutical composition of claim 11 or the method of claim 11, wherein said vascular obliteration is selected from the group consisting of thrombosis, thromboembolism and embolism.

14. The pharmaceutical composition of claim 13 or the method of claim 13, wherein said embolism is chronic pulmonary embolism.

15. The pharmaceutical composition of claim 11 or the method of claim 11, wherein said wound healing is to be stimulated upon amputation and/or transplantation of tissues and/or organs or wherein said wound healing is to be stimulated in damaged or injured blood vessels.

16. The pharmaceutical composition of claim 11 or the method of claim 11, wherein the treatment or prevention of said arterio- and veno-microangiopathy comprises the treatment of diabetes.

17. The pharmaceutical composition of claim 11 or the method of claim 11, wherein said arterio- and veno-microangiopathy is non-diabetic microangiopathy of the retina.

18. The pharmaceutical composition of claim 11 or the method of claim 11, wherein said necrosis treatment comprises the reactivation of a necrotisising tissue or of a post ischemic scar upon myocardial infarction,

19. The pharmaceutical composition of claim 11 or the method of claim 11, wherein said reactivation of hibernating tissue is reactivation of a hibernating myocardium upon myocardial infarction.

20. The pharmaceutical composition of any one of claims 1, 3 to 8 and 11 to 19 or the method of any of claims 2 to 7 and 9 to 19, wherein said compound is administered by any one of a parenteral route, topical route, oral route, intravenous route, subcutaneous route, intranasal route or transdermal route.

21. The pharmaceutical composition of any one of claims 1, 3 to 8 and 11 to 20 or the method of any of claims 2 to 7 and 9 to 20, wherein said compound is obtained from a plant belonging to the genus Leontopodium.

22. The pharmaceutical composition of claim 21 or the method of claim 21, wherein said plant is Leontopodium alpinum Cass. (Edelweiss).
